(19) United States
(12) Patent Application Publication
(10) Pub. No.: US 2021/0343411 A1
(43) Pub. Date: Nov. 4, 2021

(54) DEEP LEARNING-BASED DIAGNOSIS AND REFERRAL OF DISEASES AND DISORDERS USING NATURAL LANGUAGE PROCESSING

(71) Applicants: AI TECHNOLOGIES INC., Grand Cayman, KY (US); THE REGENTS OF THE UNIVERSITY OF CALIFORNIA, Oakland, CA (US)

(72) Inventors: Kang ZHANG, Del Mar, CA (US); Zhihuan LI, Dongguan (CN); Lianghong ZHENG, Shenyang (CN)

(21) Appl. No.: 17/136,018

(22) Filed: Dec. 29, 2020

Related U.S. Application Data
(63) Continuation of application No. PCT/US2019/039955, filed on Jun. 28, 2019.
(60) Provisional application No. 62/692,572, filed on Jun. 29, 2018.
(60) Provisional application No. 62/749,612, filed on Oct. 23, 2018.
(60) Provisional application No. 62/783,962, filed on Dec. 21, 2018.

(51) Int. Cl. G16H 50/20; G06N 3/08; G16H 10/60; G16H 50/70; G06F 40/205

(52) U.S. Cl. CPC G16H 50/20; G06N 3/08; G16H 70/20; G16H 50/70; G06F 40/205; G16H 10/60

(57) ABSTRACT

Disclosed herein are methods and systems for Artificial Intelligence (AI)-based methods for performing medical diagnosis of diseases and conditions. An automated natural language processing (NLP) system performs deep learning techniques to extract clinically relevant information from electronic health records (EHRs). This framework provides a high diagnostic accuracy that demonstrates a successful AI-based method for systematic disease diagnosis and management.

### CROSS-REFERENCE

This application is a continuation of International Application PCT/US2019/039955 filed Jun. 28, 2019, which claims the benefit of U.S. Provisional Application No. 62/692,572, filed Jun. 29, 2018, U.S. Provisional Application No. 62/749,612, filed Oct. 23, 2018, and U.S. Provisional Application No. 62/783,962, filed Dec. 21, 2018. The disclosure of each of these prior-filed applications is incorporated herein by reference in its entirety.

### BACKGROUND OF THE DISCLOSURE

Medical information has become increasingly complex over time. The range of disease entities, diagnostic testing and biomarkers, and treatment modalities has increased exponentially in recent years. Subsequently, clinical decision-making has also become more complex and demands the synthesis of numerous data points.

### SUMMARY OF THE DISCLOSURE

In the current digital age, the electronic health record (EHR) represents a massive repository of electronic data points representing a diverse array of clinical information. Disclosed herein are Artificial intelligence (AI) methods that provide powerful tools to mine and utilize EHR data for disease diagnosis and management, which can mimic and/or augment the clinical decision-making of human physicians.

To formulate a diagnosis for any given patient, physicians frequently use hypothetical deductive reasoning. Starting with the chief complaint, the physician then asks appropriately targeted questions relating to that complaint. From this initial small feature set, the physician forms a differential diagnosis and decides what feature (historical questions, physical exam findings, laboratory testing, and/or imaging studies) to obtain next to rule in or rule out the diagnoses in the differential diagnosis set. The most useful features are identified, such that when the probability of one of the diagnoses reaches a predetermined level of acceptability, the process is stopped, and the diagnosis is accepted. It may be possible to achieve an acceptable level of certainty of the diagnosis with only a few features without having to process the entire feature set. Therefore, the physician can be considered a classifier of sorts.

Described herein is an AI-based system using machine learning to extract clinically relevant features from EHR notes to mimic the clinical reasoning of human physicians. In medicine, machine learning methods have been generally limited to image-based diagnoses, but analysis of EHR data presents a number of difficult challenges. These challenges include the vast quantity of data, the use of unstructured text, the complexity of language processing, high dimensionality, data sparsity, the extent of irregularity (noise), and deviations or systematic errors in medical data. Furthermore, the same clinical phenotype can be expressed as multiple different codes and terms. These challenges make it difficult to use machine learning methods to perform accurate pattern recognition and generate predictive clinical models. Conventional approaches typically require expert knowledge and are labor-intensive, which make it difficult to scale and generalize, or are sparse, noisy, and repetitive. The machine learning methods described herein can overcome these limitations.

Described herein are systems and methods utilizing a data mining framework for EHR data that integrates prior medical knowledge and data-driven modeling. In some embodiments, an automated deep learning-based language processing system is developed and utilized to extract clinically relevant information. In some embodiments, a diagnostic system is established based on extracted clinical features. In some embodiments, this framework is applied to the diagnosis of diseases such as pediatric diseases. This approach was tested in a large pediatric population to investigate the ability of AI-based methods to automate natural language processing methods across a large number of patient records and additionally across a diverse range of conditions.

The present disclosure solves various technical problems of automating analysis and diagnosis of diseases based on EHRs. The systems and methods described herein resolve the technical challenges discussed herein by extracting semantic data using an information model, identifying clinically relevant features using deep learning-based language processing, and utilizing the features to successfully classify or diagnose diseases.

The technological solutions to the technological problem of effectively implementing computer-based algorithmic disease diagnosis using electronic health records described herein opens up the previously unrealized potential of machine learning techniques to revolutionize EHR-based analysis and diagnosis.

Disclosed herein is a method for providing a medical diagnosis, comprising: obtaining medical data; using a natural language processing (NLP) information extraction model to extract and annotate clinical features from the medical data; and analyzing at least one of the clinical features with a disease prediction classifier to generate a classification of a disease or disorder, the classification having a sensitivity of at least 80%. In some embodiments, the NLP information extraction model comprises a deep learning procedure. In some embodiments, the NLP information extraction model utilizes a standard lexicon comprising keywords representative of assertion classes. In some embodiments, the NLP information extraction model utilizes a plurality of schema, each schema comprising a feature name, anatomical location, and value. In some embodiments, the plurality of schema comprises at least one of history of present illness, physical examination, laboratory test, radiology report, and chief complaint. In some embodiments, the method comprises tokenizing the medical data for processing by the NLP information extraction model. In some embodiments, the medical data comprises an electronic health record (EHR). In some embodiments, the classification has a specificity of at least 80%. In some embodiments, the classification has an F1 score of at least 80%. In some embodiments, the clinical features are extracted in a structured format comprising data in query-answer pairs. In some embodiments, the disease prediction classifier comprises a logistic regression classifier. In some embodiments, the disease prediction classifier comprises a decision tree. In some embodiments, the classification differentiates between a serious and a non-serious condition. In some embodiments, the classification comprises at least two levels of categorization. In some embodiments, the classification comprises a first level category indicative of an organ system. In some embodiments, the classification comprises a second level indicative of a subcategory of the organ system. In some embodiments, the classification comprises a diagnostic hierarchy that categorizes the disease or disorder into a series of narrower categories. In some embodiments, the classification comprises a categorization selected from the group consisting of respiratory diseases, genitourinary diseases, gastrointestinal diseases, neuropsychiatric diseases, and systemic generalized diseases. In some embodiments, the classification further comprises a subcategorization of respiratory diseases into upper respiratory diseases and lower respiratory diseases. In some embodiments, the classification further comprises a subcategorization of upper respiratory disease into acute upper respiratory disease, sinusitis, or acute laryngitis. In some embodiments, the classification further comprises a subcategorization of lower respiratory disease into bronchitis, pneumonia, asthma, or acute tracheitis. In some embodiments, the classification further comprises a subcategorization of gastrointestinal diseases into diarrhea, mouth-related diseases, or acute pharyngitis. In some embodiments, the classification further comprises a subcategorization of neuropsychiatric diseases into tic disorder, attention-deficit hyperactivity disorder, bacterial meningitis, encephalitis, or convulsions. In some embodiments, the classification further comprises a subcategorization of systemic generalized diseases into hand, foot and mouth disease, varicella without complication, influenza, infectious mononucleosis, sepsis, or exanthema subitum. In some embodiments, the method further comprises making a medical treatment recommendation based on the classification.

Disclosed herein is non-transitory computer-readable medium comprising machine-executable code that, upon execution by one or more computer processors, implements a method for providing a classification of a disease or disorder, the method comprising: obtaining medical data; using a natural language processing (NLP) information extraction model to extract and annotate clinical features from the medical data; and analyzing at least one of the clinical features with a disease prediction classifier to generate the classification of a disease or disorder, the classification having a sensitivity of at least 80%. In some embodiments, the NLP information extraction model comprises a deep learning procedure. In some embodiments, the NLP information extraction model utilizes a standard lexicon comprising keywords representative of assertion classes. In some embodiments, the NLP information extraction model utilizes a plurality of schema, each schema comprising a feature name, anatomical location, and value. In some embodiments, the plurality of schema comprises at least one of history of present illness, physical examination, laboratory test, radiology report, and chief complaint. In some embodiments, the method comprises tokenizing the medical data for processing by the NLP information extraction model. In some embodiments, the medical data comprises an electronic health record (EHR). In some embodiments, the classification has a specificity of at least 80%. In some embodiments, the classification has an F1 score of at least 80%. In some embodiments, the clinical features are extracted in a structured format comprising data in query-answer pairs. In some embodiments, the disease prediction classifier comprises a logistic regression classifier. In some embodiments, the disease prediction classifier comprises a decision tree. In some embodiments, the classification differentiates between a serious and a non-serious condition. In some embodiments, the classification comprises at least two levels of categorization. In some embodiments, the classification comprises a first level category indicative of an organ system. In some embodiments, the classification comprises a second level indicative of a subcategory of the organ system. In some embodiments, the classification comprises a diagnostic hierarchy that categorizes the disease or disorder into a series of narrower categories. In some embodiments, the classification comprises a categorization selected from the group consisting of respiratory diseases, genitourinary diseases, gastrointestinal diseases, neuropsychiatric diseases, and systemic generalized diseases. In some embodiments, the classification further comprises a subcategorization of respiratory diseases into upper respiratory diseases and lower respiratory diseases. In some embodiments, the classification further comprises a subcategorization of upper respiratory disease into acute upper respiratory disease, sinusitis, or acute laryngitis. In some embodiments, the classification further comprises a subcategorization of lower respiratory disease into bronchitis, pneumonia, asthma, or acute tracheitis. In some embodiments, the classification further comprises a subcategorization of gastrointestinal diseases into diarrhea, mouth-related diseases, or acute pharyngitis. In some embodiments, the classification further comprises a subcategorization of neuropsychiatric diseases into tic disorder, attention-deficit hyperactivity disorder, bacterial meningitis, encephalitis, or convulsions. In some embodiments, the classification further comprises a subcategorization of systemic generalized diseases into hand, foot and mouth disease, varicella without complication, influenza, infectious mononucleosis, sepsis, or exanthema subitum. In some embodiments, the method further comprises making a medical treatment recommendation based on the classification.

Disclosed herein is a computer-implemented system comprising: a digital processing device comprising: at least one processor, an operating system configured to perform executable instructions, a memory, and a computer program including instructions executable by the digital processing device to create an application for providing a medical diagnosis, the application comprising: a software module obtaining medical data; a software module using a natural language processing (NLP) information extraction model to extract and annotate clinical features from the medical data; and a software module analyzing at least one of the clinical features with a disease prediction classifier to generate the classification of a disease or disorder, the classification having a sensitivity of at least 80%. In some embodiments, the NLP information extraction model comprises a deep learning procedure. In some embodiments, the NLP information extraction model utilizes a standard lexicon comprising keywords representative of assertion classes. In some embodiments, the NLP information extraction model utilizes a plurality of schema, each schema comprising a feature name, anatomical location, and value. In some embodiments, the plurality of schema comprises at least one of history of present illness, physical examination, laboratory test, radiology report, and chief complaint. In some embodiments, the system further comprises a software module tokenizing the medical data for processing by the NLP information extraction model. In some embodiments, the medical data comprises an electronic health record (EHR). In some embodiments, the classification has a specificity of at least 80%. In some embodiments, the classification has an F1 score of at least 80%. In some embodiments, the clinical features are extracted in a structured format comprising data in query-answer pairs. In some embodiments, the disease prediction classifier comprises a logistic regression classifier. In some embodiments, the disease prediction classifier comprises a decision tree. In some embodiments, the classification differentiates between a serious and a non-serious condition. In some embodiments, the classification comprises at least two levels of categorization. In some embodiments, the classification comprises a first level category indicative of an organ system. In some embodiments, the classification comprises a second level indicative of a subcategory of the organ system. In some embodiments, the classification comprises a diagnostic hierarchy that categorizes the disease or disorder into a series of narrower categories. In some embodiments, the classification comprises a categorization selected from the group consisting of respiratory diseases, genitourinary diseases, gastrointestinal diseases, neuropsychiatric diseases, and systemic generalized diseases. In some embodiments, the classification further comprises a subcategorization of respiratory diseases into upper respiratory diseases and lower respiratory diseases. In some embodiments, the classification further comprises a subcategorization of upper respiratory disease into acute upper respiratory disease, sinusitis, or acute laryngitis. In some embodiments, the classification further comprises a subcategorization of lower respiratory disease into bronchitis, pneumonia, asthma, or acute tracheitis. In some embodiments, the classification further comprises a subcategorization of gastrointestinal diseases into diarrhea, mouth-related diseases, or acute pharyngitis. In some embodiments, the classification further comprises a subcategorization of neuropsychiatric diseases into tic disorder, attention-deficit hyperactivity disorder, bacterial meningitis, encephalitis, or convulsions. In some embodiments, the classification further comprises a subcategorization of systemic generalized diseases into hand, foot and mouth disease, varicella without complication, influenza, infectious mononucleosis, sepsis, or exanthema subitum. In some embodiments, the method further comprises making a medical treatment recommendation based on the classification.

In another aspect, disclosed herein is a computer-implemented method for generating a disease prediction classifier for providing a medical diagnosis, comprising: a) providing a lexicon constructed based on medical texts, wherein the lexicon comprises keywords relating to clinical information; b) obtaining medical data comprising electronic health records (EHRs); c) extracting clinical features from the medical data using an NLP information extraction model; d) mapping the clinical features to hypothetical clinical queries to generate question-answer pairs; and e) training the NLP classifier using the question-answer pairs, wherein the NLP classifier is configured to generate classifications having a sensitivity of at least 80% when tested against an independent dataset of at least 100 EHRs. In some embodiments, the NLP information extraction model comprises a deep learning procedure. In some embodiments, the NLP information extraction model utilizes a standard lexicon comprising keywords representative of assertion classes. In some embodiments, the NLP information extraction model utilizes a plurality of schema, each schema comprising a feature name, anatomical location, and value. In some embodiments, the plurality of schema comprises at least one of history of present illness, physical examination, laboratory test, radiology report, and chief complaint. In some embodiments, the method comprises tokenizing the medical data for processing by the NLP information extraction model. In some embodiments, the medical data comprises an electronic health record (EHR). In some embodiments, the classification has a specificity of at least 80%. In some embodiments, the classification has an F1 score of at least 80%. In some embodiments, the clinical features are extracted in a structured format comprising data in query-answer pairs. In some embodiments, the disease prediction classifier comprises a logistic regression classifier. In some embodiments, the disease prediction classifier comprises a decision tree. In some embodiments, the classification differentiates between a serious and a non-serious condition. In some embodiments, the classification comprises at least two levels of categorization. In some embodiments, the classification comprises a first level category indicative of an organ system. In some embodiments, the classification comprises a second level indicative of a subcategory of the organ system. In some embodiments, the classification comprises a diagnostic hierarchy that categorizes the disease or disorder into a series of narrower categories. In some embodiments, the classification comprises a categorization selected from the group consisting of respiratory diseases, genitourinary diseases, gastrointestinal diseases, neuropsychiatric diseases, and systemic generalized diseases. In some embodiments, the classification further comprises a subcategorization of respiratory diseases into upper respiratory diseases and lower respiratory diseases. In some embodiments, the classification further comprises a subcategorization of upper respiratory disease into acute upper respiratory disease, sinusitis, or acute laryngitis. In some embodiments, the classification further comprises a subcategorization of lower respiratory disease into bronchitis, pneumonia, asthma, or acute tracheitis. In some embodiments, the classification further comprises a subcategorization of gastrointestinal diseases into diarrhea, mouth-related diseases, or acute pharyngitis. In some embodiments, the classification further comprises a subcategorization of neuropsychiatric diseases into tic disorder, attention-deficit hyperactivity disorder, bacterial meningitis, encephalitis, or convulsions. In some embodiments, the classification further comprises a subcategorization of systemic generalized diseases into hand, foot and mouth disease, varicella without complication, influenza, infectious mononucleosis, sepsis, or exanthema subitum. In some embodiments, the method further comprises making a medical treatment recommendation based on the classification.

In another aspect, disclosed herein is a non-transitory computer-readable medium comprising machine-executable code that, upon execution by one or more computer processors, implements a method for generating a natural language processing (NLP) classifier for providing a classification of a disease or disorder, the method comprising: a) providing a lexicon constructed based on medical texts, wherein the lexicon comprises keywords relating to clinical information; b) obtaining medical data comprising electronic health records (EHRs); c) extracting clinical features from the medical data using an NLP information extraction model; d) mapping the clinical features to hypothetical clinical queries to generate question-answer pairs; and e) training the NLP classifier using the question-answer pairs, wherein the NLP classifier is configured to generate classifications having a sensitivity of at least 80% when tested against an independent dataset of at least 100 EHRs. In some embodiments, the NLP information extraction model comprises a deep learning procedure. In some embodiments, the NLP information extraction model utilizes a standard lexicon comprising keywords representative of assertion classes. In some embodiments, the NLP information extraction model utilizes a plurality of schema, each schema comprising a feature name, anatomical location, and value. In some embodiments, the plurality of schema comprises at least one of history of present illness, physical examination, laboratory test, radiology report, and chief complaint. In some embodiments, the method comprises tokenizing the medical data for processing by the NLP information extraction model. In some embodiments, the medical data comprises an electronic health record (EHR). In some embodiments, the classification has a specificity of at least 80%. In some embodiments, the classification has an F1 score of at least 80%. In some embodiments, the clinical features are extracted in a structured format comprising data in query-answer pairs. In some embodiments, the disease prediction classifier comprises a logistic regression classifier. In some embodiments, the disease prediction classifier comprises a decision tree. In some embodiments, the classification differentiates between a serious and a non-serious condition. In some embodiments, the classification comprises at least two levels of categorization. In some embodiments, the classification comprises a first level category indicative of an organ system. In some embodiments, the classification comprises a second level indicative of a subcategory of the organ system. In some embodiments, the classification comprises a diagnostic hierarchy that categorizes the disease or disorder into a series of narrower categories. In some embodiments, the classification comprises a categorization selected from the group consisting of respiratory diseases, genitourinary diseases, gastrointestinal diseases, neuropsychiatric diseases, and systemic generalized diseases. In some embodiments, the classification further comprises a subcategorization of respiratory diseases into upper respiratory diseases and lower respiratory diseases. In some embodiments, the classification further comprises a subcategorization of upper respiratory disease into acute upper respiratory disease, sinusitis, or acute laryngitis. In some embodiments, the classification further comprises a subcategorization of lower respiratory disease into bronchitis, pneumonia, asthma, or acute tracheitis. In some embodiments, the classification further comprises a subcategorization of gastrointestinal diseases into diarrhea, mouth-related diseases, or acute pharyngitis. In some embodiments, the classification further comprises a subcategorization of neuropsychiatric diseases into tic disorder, attention-deficit hyperactivity disorder, bacterial meningitis, encephalitis, or convulsions. In some embodiments, the classification further comprises a subcategorization of systemic generalized diseases into hand, foot and mouth disease, varicella without complication, influenza, infectious mononucleosis, sepsis, or exanthema subitum. In some embodiments, the method further comprises making a medical treatment recommendation based on the classification.

In another aspect, disclosed herein is a computer-implemented system comprising: a digital processing device comprising: at least one processor, an operating system configured to perform executable instructions, a memory, and a computer program including instructions executable by the digital processing device to create an application for generating a disease prediction classifier for providing a medical diagnosis, the application comprising: a) a software module for providing a lexicon constructed based on medical texts, wherein the lexicon comprises keywords relating to clinical information; b) a software module for obtaining medical data comprising electronic health records (EHRs); c) a software module for extracting clinical features from the medical data using an NLP information extraction model; d) a software module for mapping the clinical features to hypothetical clinical queries to generate question-answer pairs; and e) a software module for training the NLP classifier using the question-answer pairs, wherein the NLP classifier is configured to generate classifications having a sensitivity of at least 80% when tested against an independent dataset of at least 100 EHRs. In some embodiments, the NLP information extraction model comprises a deep learning procedure. In some embodiments, the NLP information extraction model utilizes a standard lexicon comprising keywords representative of assertion classes. In some embodiments, the NLP information extraction model utilizes a plurality of schema, each schema comprising a feature name, anatomical location, and value. In some embodiments, the plurality of schema comprises at least one of history of present illness, physical examination, laboratory test, radiology report, and chief complaint. In some embodiments, the method comprises tokenizing the medical data for processing by the NLP information extraction model. In some embodiments, the medical data comprises an electronic health record (EHR). In some embodiments, the classification has a specificity of at least 80%. In some embodiments, the classification has an F1 score of at least 80%. In some embodiments, the clinical features are extracted in a structured format comprising data in query-answer pairs. In some embodiments, the disease prediction classifier comprises a logistic regression classifier. In some embodiments, the disease prediction classifier comprises a decision tree. In some embodiments, the classification differentiates between a serious and a non-serious condition. In some embodiments, the classification comprises at least two levels of categorization. In some embodiments, the classification comprises a first level category indicative of an organ system. In some embodiments, the classification comprises a second level indicative of a subcategory of the organ system. In some embodiments, the classification comprises a diagnostic hierarchy that categorizes the disease or disorder into a series of narrower categories. In some embodiments, the classification comprises a categorization selected from the group consisting of respiratory diseases, genitourinary diseases, gastrointestinal diseases, neuropsychiatric diseases, and systemic generalized diseases. In some embodiments, the classification further comprises a subcategorization of respiratory diseases into upper respiratory diseases and lower respiratory diseases. In some embodiments, the classification further comprises a subcategorization of upper respiratory disease into acute upper respiratory disease, sinusitis, or acute laryngitis. In some embodiments, the classification further comprises a subcategorization of lower respiratory disease into bronchitis, pneumonia, asthma, or acute tracheitis. In some embodiments, the classification further comprises a subcategorization of gastrointestinal diseases into diarrhea, mouth-related diseases, or acute pharyngitis. In some embodiments, the classification further comprises a subcategorization of neuropsychiatric diseases into tic disorder, attention-deficit hyperactivity disorder, bacterial meningitis, encephalitis, or convulsions. In some embodiments, the classification further comprises a subcategorization of systemic generalized diseases into hand, foot and mouth disease, varicella without complication, influenza, infectious mononucleosis, sepsis, or exanthema subitum. In some embodiments, the method further comprises making a medical treatment recommendation based on the classification.

In another aspect, disclosed herein is a digital processing device comprising: at least one processor, an operating system configured to perform executable instructions, a memory, and a computer program including instructions executable by the digital processing device to create an application for generating a disease prediction classifier for providing a medical diagnosis, the application comprising: a) a software module for providing a lexicon constructed based on medical texts, wherein the lexicon comprises keywords relating to clinical information; b) a software module for obtaining medical data comprising electronic health records (EHRs); c) a software module for extracting clinical features from the medical data using an NLP information extraction model; d) a software module for mapping the clinical features to hypothetical clinical queries to generate question-answer pairs; and e) a software module for training the NLP classifier using the question-answer pairs, wherein the NLP classifier is configured to generate classifications having a sensitivity of at least 80% when tested against an independent dataset of at least 100 EHRs. In some embodiments, the NLP information extraction model comprises a deep learning procedure. In some embodiments, the NLP information extraction model utilizes a standard lexicon comprising keywords representative of assertion classes. In some embodiments, the NLP information extraction model utilizes a plurality of schema, each schema comprising a feature name, anatomical location, and value. In some embodiments, the plurality of schema comprises at least one of history of present illness, physical examination, laboratory test, radiology report, and chief complaint. In some embodiments, the method comprises tokenizing the medical data for processing by the NLP information extraction model. In some embodiments, the medical data comprises an electronic health record (EHR). In some embodiments, the classification has a specificity of at least 80%. In some embodiments, the classification has an F1 score of at least 80%. In some embodiments, the clinical features are extracted in a structured format comprising data in query-answer pairs. In some embodiments, the disease prediction classifier comprises a logistic regression classifier. In some embodiments, the disease prediction classifier comprises a decision tree. In some embodiments, the classification differentiates between a serious and a non-serious condition. In some embodiments, the classification comprises at least two levels of categorization. In some embodiments, the classification comprises a first level category indicative of an organ system. In some embodiments, the classification comprises a second level indicative of a subcategory of the organ system. In some embodiments, the classification comprises a diagnostic hierarchy that categorizes the disease or disorder into a series of narrower categories. In some embodiments, the classification comprises a categorization selected from the group consisting of respiratory diseases, genitourinary diseases, gastrointestinal diseases, neuropsychiatric diseases, and systemic generalized diseases. In some embodiments, the classification further comprises a subcategorization of respiratory diseases into upper respiratory diseases and lower respiratory diseases. In some embodiments, the classification further comprises a subcategorization of upper respiratory disease into acute upper respiratory disease, sinusitis, or acute laryngitis. In some embodiments, the classification further comprises a subcategorization of lower respiratory disease into bronchitis, pneumonia, asthma, or acute tracheitis. In some embodiments, the classification further comprises a subcategorization of gastrointestinal diseases into diarrhea, mouth-related diseases, or acute pharyngitis. In some embodiments, the classification further comprises a subcategorization of neuropsychiatric diseases into tic disorder, attention-deficit hyperactivity disorder, bacterial meningitis, encephalitis, or convulsions. In some embodiments, the classification further comprises a subcategorization of systemic generalized diseases into hand, foot and mouth disease, varicella without complication, influenza, infectious mononucleosis, sepsis, or exanthema subitum. In some embodiments, the method further comprises making a medical treatment recommendation based on the classification.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the present invention will be obtained by reference to the following detailed description that sets forth illustrative embodiments, in which the principles of the invention are utilized, and the accompanying drawings of which:

FIG. 1 shows the results of unsupervised clustering of pediatric diseases.

FIG. 2 shows an example of a workflow diagram for data extraction, analysis, and diagnosis.

FIG. 3 shows an example of a hierarchy of the diagnostic framework in a large pediatric cohort.

FIG. 4 shows a flow chart illustrating extraction of relevant information from an input EHR sentence segment to generate question-answer query-answer pairs using a LSTM model.

FIG. 5 shows a workflow diagram that depicts an embodiment of the hybrid natural language processing and machine learning AI-based system.

FIGS. 6A-6D shows the diagnostic efficiencies and model performance for GMU1 adult data and GWCMC1 pediatric data. FIG. 6A shows a convolutional table showing diagnostic efficiencies across adult populations. FIG. 6B shows an ROC-AUC curve for model performance across adult populations. FIG. 6C shows a convolutional table showing diagnostic efficiencies across pediatric populations. FIG. 6D shows an ROC-AUC curve for model performance across pediatric populations.

FIGS. 7A-7D shows the diagnostic efficiencies and model performance for GMU2 adult data and GWCMC2 pediatric data. FIG. 7A shows a convolutional table showing diagnostic efficiencies across adult populations. FIG. 7B shows an ROC-AUC curve for model performance across adult populations. FIG. 7C shows a convolutional table showing diagnostic efficiencies across pediatric populations. FIG. 7D shows an ROC-AUC curve for model performance across pediatric populations.

FIGS. 8A-8F shows Comparison of Hierarchical Diagnosis Approach (right) versus end-to-end approach in pediatric respiratory diseases (left). FIGS. 8A-8C shows an end-to-end approach. FIG. 8A depicts a confusion table showing diagnostic efficiencies between upper and lower respiratory systems in pediatric patients. FIG. 8B depicts a confusion table showing diagnostic efficiencies in top four upper-respiratory diseases. FIG. 8C shows a confusion table showing diagnostic efficiencies in top six lower-respiratory diseases. FIGS. 8D-8F show a hierarchical diagnostic approach. FIG. 8D depicts a confusion table showing diagnostic efficiencies for upper and lower respiratory systems in pediatric patients. FIG. 8E depicts a confusion table showing diagnostic efficiencies in top four upper-respiratory diseases. FIG. 8F depicts a confusion table showing diagnostic efficiencies in top six lower-respiratory diseases.

FIG. 9 shows an example of free-text document record of an endocrinological and metabolic disease case that can be used in segmentation.

FIG. 10A, FIG. 10B, FIG. 10C, and FIG. 10D show model performance over time with percent classification and loss over number of epochs in adult and pediatric internal validations.

### DETAILED DESCRIPTION OF THE DISCLOSURE

It is recognized that implementation of clinical decision support algorithms for medical imaging with improved reliability and clinical interpretability can be achieved through one or combinations of technical features of the present disclosure. According to some aspects, disclosed herein is a diagnostic tool to correctly identify diseases or disorders by presenting a machine learning framework developed for diseases or conditions such as common and dangerous pediatric disorders. In some embodiments, the machine learning framework utilizes deep learning models such as artificial neural networks. In some embodiments, the model disclosed herein generalizes and performs well on many medical classification tasks. This framework can be applied towards medical data such as electronic health records. Certain embodiments of this approach yield superior performance across many types of medical records.

### Medical Data

In certain aspects, the machine learning framework disclosed herein is used for analyzing medical data. In some embodiments, the medical data comprises electronic health records (EHRs). In some embodiments, an EHR is a digital version of a paper chart used in a clinician's office. In some embodiments, an EHR comprises the medical and treatment history of a patient. In some embodiments, an EHR allows patient data to be tracked over time.

In some embodiments, medical data comprises patient information such as identifying information, age, sex or gender, race or ethnicity, weight, height, body mass index (BMI), heart rate (e.g. ECG and/or peripheral pulse rate), blood pressure, body temperature, respiration rate, past checkups, treatments or therapies, drugs administered, observations, vaccinations, current and/or past symptoms (e.g. fever, vomiting, cough, etc.), known health conditions (e.g. allergies), known diseases or disorders, health history (e.g. past diagnoses), lab test results (e.g. blood test), lab imaging results (e.g. X-rays, MRIs, etc.), genetic information (e.g. known genetic abnormalities associated with disease), family medical history, or any combination thereof. The framework described herein is applicable to various types of medical data in addition to EHRs.

### Machine Learning

In certain aspects, disclosed herein are machine learning frameworks for generating models or classifiers that diagnose, predict, or classify one or more disorders or conditions. In some embodiments, disclosed herein is a classifier diagnosing one or more disorders or conditions based on medical data such as an electronic health record (EHR). In some embodiments, the medical data comprises one or more clinical features entered or uploaded by a user. In some embodiments, the classifier exhibits higher sensitivity, specificity, and/or AUC for an independent sample set compared to an average human clinician (e.g. an average clinician). In some embodiments, the classifier provides a sensitivity (true positive rate) of at least about 0.7, about 0.75, about 0.8, about 0.85, about 0.9, about 0.95, or about 0.99 and/or a specificity (true negative rate) of at least about 0.7, about 0.75, about 0.8, about 0.85, about 0.9, about 0.95, or about 0.99 when tested against at least 100, 200, 300, 400, 500, 600, 700, 800, 900, or 1000 independent samples (e.g. an EHR or medical data entered by a clinician). In some embodiments, the classifier has an AUC of at least about 0.7, about 0.75, about 0.8, about 0.85, about 0.9, about 0.95, or about 0.99 when tested against at least 100, 200, 300, 400, 500, 600, 700, 800, 900, or 1000 independent samples.

Various algorithms can be used to generate models that generate a prediction based on the input data (e.g., EHR information). In some instances, machine learning methods are applied to the generation of such models (e.g. trained classifier). In some embodiments, the model is generated by providing a machine learning algorithm with training data in which the expected output is known in advance.

In some embodiments, the systems, devices, and methods described herein generate one or more recommendations such as treatment and/or healthcare options for a subject. In some embodiments, the one or more treatment recommendations are provided in addition to a diagnosis or detection of a disease or condition. In some embodiments, a treatment recommendation is a recommended treatment according to standard medical guidelines for the diagnosed disease or condition. In some embodiments, the systems, devices, and methods herein comprise a software module providing one or more recommendations to a user. In some embodiments, the treatment and/or healthcare option are specific to the diagnosed disease or condition.

In some embodiments, a classifier or trained machine learning algorithm of the present disclosure comprises a feature space. In some cases, the classifier comprises two or more feature spaces. The two or more feature spaces may be distinct from one another. In some embodiments, a feature space comprises information such as formatted and/or processed EHR data. When training the machine learning algorithm, training data such as EHR data is input into the algorithm which processes the input features to generate a model. In some embodiments, the machine learning algorithm is provided with training data that includes the classification (e.g., diagnostic or test result), thus enabling the algorithm to train by comparing its output with the actual output to modify and improve the model. This is often referred to as supervised learning. Alternatively, in some embodiments, the machine learning algorithm can be provided with unlabeled or unclassified data, which leaves the algorithm to identify hidden structure amongst the cases (referred to as unsupervised learning). Sometimes, unsupervised learning is useful for identifying the features that are most useful for classifying raw data into separate cohorts.

In some embodiments, one or more sets of training data are used to train a machine learning algorithm. Although exemplar embodiments of the present disclosure include machine learning algorithms that use convolutional neural networks, various types of algorithms are contemplated. In some embodiments, the algorithm utilizes a predictive model such as a neural network, a decision tree, a support vector machine, or other applicable model. In some embodiments, the machine learning algorithm is selected from the group consisting of a supervised, semi-supervised and unsupervised learning, such as, for example, a support vector machine (SVM), a Naïve Bayes classification, a random forest, an artificial neural network, a decision tree, a K-means, learning vector quantization (LVQ), self-organizing map (SOM), graphical model, regression algorithm (e.g., linear, logistic, multivariate, association rule learning, deep learning, dimensionality reduction and ensemble selection algorithms. In some embodiments, the machine learning algorithm is selected from the group consisting of: a support vector machine (SVM), a Naïve Bayes classification, a random forest, and an artificial neural network. Machine learning techniques include bagging procedures, boosting procedures, random forest algorithms, and combinations thereof. Illustrative algorithms for analyzing the data include but are not limited to methods that handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis.

### Unsupervised Diagnostic Grouping

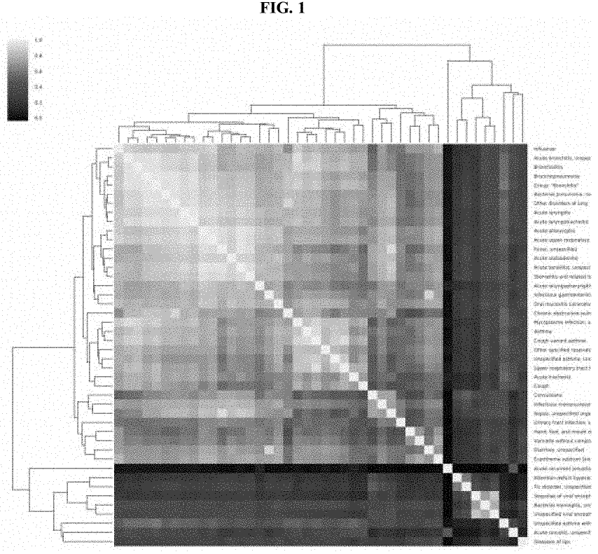

Disclosed herein are systems and methods utilizing unsupervised clustering to identify trends in clinical features. In some embodiments, the EHR(s) are analyzed in the absence of a defined classification system with human input. In some embodiments, trends in clinical features were detected in the absence of pre-defined labeling in order to generate a grouping structure such as shown in FIG. 1. In some embodiments, at least some of the diagnoses that were clustered together had related ICD-10 codes. This reflects the ability to detect trends in clinical features that align with a human-defined classification system. In some embodiments, at least some of the related diagnoses (e.g. based on ICD-10 codes) were clustered together, but did not include other similar diagnoses within this cluster.

### Medical Record Reformatting Using Natural Language Processing

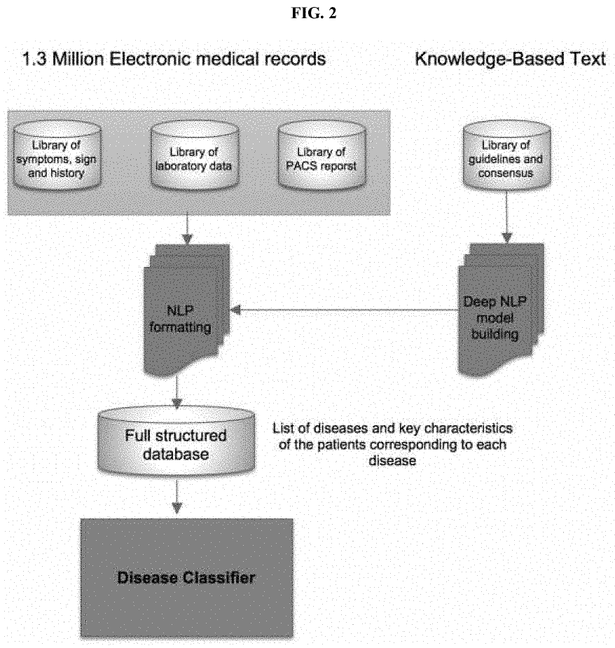

Disclosed herein are systems and methods utilizing natural language processing to extract the key concepts and/or features from medical data. In some embodiments, the NLP framework comprises at least one of the following: 1) lexicon construction, 2) tokenization, 3) word embedding, 4) schema construction, and 5) sentence classification using Long Short Term Memory (LSTM) architecture. In some embodiments, medical charts are manually annotated using a schema. In some embodiments, the annotated charts are used to train a NLP information extraction model. In some embodiments, a subset of the annotated charts are withheld from the training set and used to validate the model. In some embodiments, the information extraction model summarized the key conceptual categories representing clinical data (FIG. 2). In some embodiments, the NLP model utilizes deep learning techniques to automate the annotation of the free text EHR notes into a standardized lexicon. In some embodiments, the NLP model allows further processing of the standardized data for diagnostic classification.

In some embodiments, an information extraction model was generated for summarizing the key concepts and associated categories used in representing reformatted clinical data (Supplementary Table 1). In some embodiments, the reformatted chart groups the relevant symptoms into categories. This has the benefit of increased transparency by showing the exact features that the model relies on to make a diagnosis. In some embodiments, the schemas are curated and validated by physician(s) and/or medical experts. In some embodiments, the schemas include at least one of chief complaint, history of present illness, physical examination, and lab reports.

Lexicon Construction

In some embodiments, an initial lexicon is developed based on history of present illness (HPI) narratives presented in standard medical texts. In some embodiments, the lexicon is enriched by manually reading sentences in the training data (e.g. 1% of each class, consisting of over 11,967 sentences) and selecting words representative of the assertion classes. In some embodiments, the keywords are curated by physicians. In some embodiments, the keywords are optionally generated by using a medical dictionary such as the Chinese medical dictionary (e.g. the Unified Medical Language System, or UMLS16). In some embodiments, the errors in the lexicon are revised according to physicians' clinical knowledge and experience, as well as expert consensus guidelines. In some embodiments, the lexicon is revised based on information derived from board-certified internal medicine physicians, informaticians, health information management professionals, or any combination thereof. In some embodiments, this procedure is iteratively conducted until no new concepts of HPI and PE are found.

Schema Design

In some embodiments, an information schema is a rule-based synthesis of medical knowledge and/or physician experience. In some embodiments, once the schema is fixed, the information that natural language processing can obtain from the medical records is also fixed. In some embodiments, schema comprises question-and-answer pairs. In some embodiments, the question-and-answer pairs are physician curated. In some embodiments, the curated question-and-answer pairs are used by the physician(s) in extracting symptom information towards making a diagnosis. Examples of questions are the following: Is patient having a fever?, Is the patient coughing?, etc. The answer consists of a key_location and a numeric feature. The key_location encodes anatomical locations such as lung, gastrointestinal tract, etc.

In some embodiments, the value is either a categorical variable or a binary number depending on the feature type. In some embodiments, a schema is constructed for each type of medical record data such as, for example, the history of present illness and chief complaint, physical examination, laboratory tests, and radiology reports. In some embodiments, this schema is applied towards the text re-formatting model construction.

One advantage for this schema design is the increase or maximization of data interoperability across hospitals for future study. The pre-defined space of query-answers pairs simplifies the data interpolation process across EHR systems from multiple hospitals. Also, providing clinical information in reduced formats can help protect patient privacy compared to providing raw clinical notes which could be patient-identifiable. Even with removal of patient-identifiable variables, the style of writing in the EHR may potentially reveal the identity of the examining physician, as suggested by advances in stylometry tools, which could increase patient identifiability

In some embodiments, a schema comprises a group of items. In some embodiments, a schema comprises three items <item_name, key_location, value>. In some embodiments, the item_name is the feature name. In some embodiments, the key_location encodes anatomical locations. In some embodiments, the value consists of either free text or a binary number depending on the query type. In some embodiments, when doing pattern matching, the NLP results are assessed to check if they could match to certain schema, and the results are filled out to the fourth column of the form while the first three columns remained unchanged unchanged.

In some embodiments, a schema is constructed with the curation of physicians. In some embodiments, a schema is selected from: history of present illness, physical examination, laboratory tests, and radiology reports. In some embodiments, the chief complaint and history of present illness shared the same schema. Non-limiting embodiments of information schema are shown in Supplementary Table 1.

Tokenization and Word Embedding

In some embodiments, standard datasets for word segmentation are generated. This provides a solution to any lack of publicly available community annotated resources. In some embodiments, the tool used for tokenization is mecab (url: https://github.com/taku910/mecab), with the curated lexicons described herein as the optional parameter. In some embodiments, a minimum number of tokens are generated for use in the NLP framework. In some embodiments, a maximum number of tokens are generated for use in the NLP framework. In some embodiments, the NLP framework utilizes at least 500 tokens, at least 1000 tokens, at least 2000 tokens, at least 3000 tokens, at least 4000 tokens, at least 5000 tokens, at least 6000 tokens, at least 7000 tokens, at least 8000 tokens, at least 9000 tokens, or at least 10000 tokens or more. In some embodiments, the NLP framework utilizes no more than 500 tokens, no more than 1000 tokens, no more than 2000 tokens, no more than 3000 tokens, no more than 4000 tokens, no more than 5000 tokens, no more than 6000 tokens, no more than 7000 tokens, no more than 8000 tokens, no more than 9000 tokens, or no more than 10000 tokens. In some embodiments, the NLP framework described herein utilizes a number of features. In some embodiments, the features are high dimensional features. In some embodiments, the tokens are embedded with features. In some embodiments, the tokens are embedded with at least 10 features, at least 20 features, at least 30 features, at least 40 features, at least 50 features, at least 60 features, at least 70 features, at least 80 features, at least 90 features, at least 100 features, at least 120 features, at least 140 features, at least 160 features, at least 180 features, at least 200 features, at least 250 features, at least 300 features, at least 400 features, or at least 500 features. For example, word2vec from python Tensorflow package was used to embed 4363 tokens with 100 high dimensional features.

LSTM Model Training Data Set and Testing Data Set Construction

In some embodiments, a data set is curated for training the text classification model. In some embodiments, the query-answer pairs in the training and validation cohort are manually annotated. In some embodiments, the training data set comprises at least 500, at least 1000, at least 1500, at least 2000, at least 2500, at least 3000, at least 3500, at least 4000, at least 4500, at least 5000, at least 6000, at least 7000, at least 8000, at least 9000, or at least 10000 query-answer pairs. In some embodiments, the training data set comprises no more than 500, no more than 1000, no more than 1500, no more than 2000, no more than 2500, no more than 3000, no more than 3500, no more than 4000, no more than 4500, no more than 5000, no more than 6000, no more than 7000, no more than 8000, no more than 9000, or no more than 10000 query-answer pairs. In some embodiments, for questions with binary answers, 0/1 is used to indicate if the text gave a no/yes. For example, given the text snippet “patient has fever”, query “is patient having fever?” can be assigned a value of 1. In some embodiments, for queries with categorical/numerical values, the pre-defined categorical free text answer is extracted as shown in the schema (Supplementary Table 1).

In some embodiments, the free-text harmonization process is modeled by an attention-based LSTM. In some embodiments, the model is implemented using tensorflow and trained with a number of steps. In some embodiments, the number of steps is at least 50,000, at least 75,000 steps, at least 100,000 steps, at least 125,000 steps, at least 150,000 steps, at least 175,000 steps, at least 200,000 steps, at least 250,000 steps, at least 300,000 steps, at least 400,000 steps, or at least 500,000 steps. In some embodiments, the number of steps is no more than 50,000, no more than 75,000 steps, no more than 100,000 steps, no more than 125,000 steps, no more than 150,000 steps, no more than 175,000 steps, no more than 200,000 steps, no more than 250,000 steps, no more than 300,000 steps, no more than 400,000 steps, or no more than 500,000 steps. In some embodiments, the NLP model is applied to physician notes, which have been converted into the structured format, where each structured record contained data in query-answer pairs.

A non-limiting embodiment of the NLP model demonstrates excellent results in annotation of the EHR physician notes (see Table 2 in Example 1). Across all categories of clinical data (chief complaint, history of present illness, physical examination, laboratory testing, and PACS reports), the F1 score exceeded 90% except in one instance, which was for categorical variables detected in laboratory testing. The recall of the NLP model was highest for physical examination (95.62% for categorical variables, 99.08% for free text), and lowest for laboratory testing (72.26% for categorical variables, 88.26% for free text). The precision of the NLP model was highest for chief complaint (97.66% for categorical variables, 98.71% for free text), and lowest for laboratory testing (93.78% for categorical variables, and 96.67% for free text). In general, the precision (or positive predictive value) of the NLP labeling was slightly greater than the recall (the sensitivity), but the system demonstrated overall strong performance across all domains.

In some embodiments, the NLP model produces annotation of the medical data sample (e.g. EHR physician notes) with a performance measured by certain metrics such as recall, precision, F1 score, and/or instances of exact matches for each category of clinical data. In some embodiments, the NLP model has an F1 score of at least 70%, at least 75%, at least 80%, at least 85%, at least 90%, at least 91%, at least 92%, at least 93%, at least 94%, at least 95%, at least 96%, at least 97%, at least 98%, or at least 99% for at least one category of clinical data. In some embodiments, the NLP model produces a recall of at least 70%, at least 75%, at least 80%, at least 85%, at least 90%, at least 91%, at least 92%, at least 93%, at least 94%, at least 95%, at least 96%, at least 97%, at least 98%, or at least 99% for at least one category of clinical data. In some embodiments, the NLP model produces a precision of at least 70%, at least 75%, at least 80%, at least 85%, at least 90%, at least 91%, at least 92%, at least 93%, at least 94%, at least 95%, at least 96%, at least 97%, at least 98%, or at least 99% for at least one category of clinical data. In some embodiments, the NLP model produces an exact match of at least 70%, at least 75%, at least 80%, at least 85%, at least 90%, at least 91%, at least 92%, at least 93%, at least 94%, at least 95%, at least 96%, at least 97%, at least 98%, or at least 99% for at least one category of clinical data. In some embodiments, the at least one category of clinical data comprises chief complaint, history of present illness, physical examination, laboratory testing, PACS report, or any combination thereof. In some embodiments, a category of clinical data comprises a classification, categorical variable(s), free text, or any combination thereof

Performance of the Model in Diagnostic Accuracy

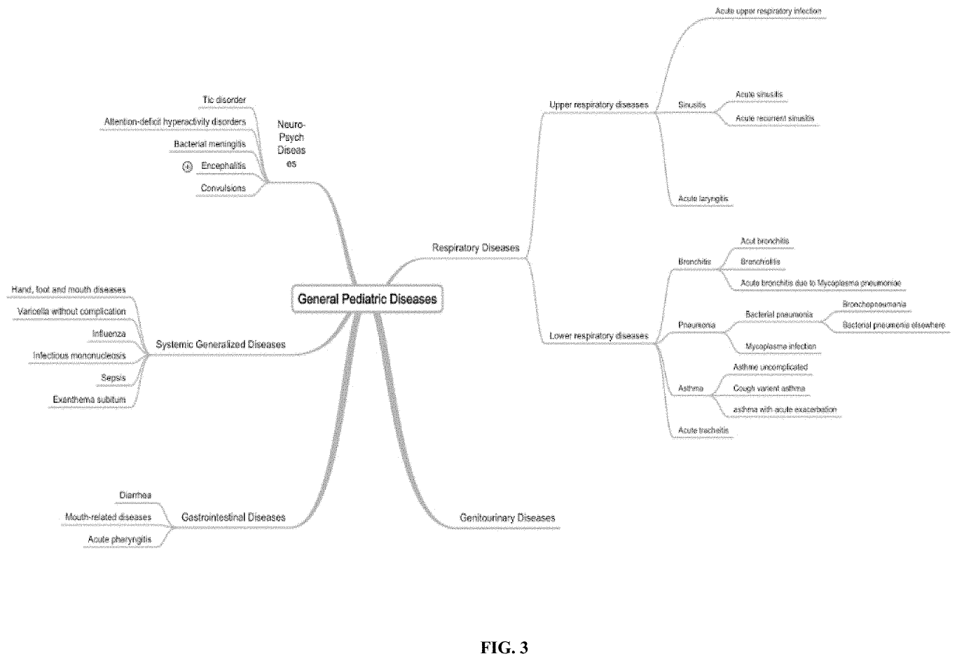

In some embodiments, after annotation of the EHR notes, a logistic regression classifier is used to establish a diagnostic system (FIG. 3). In some embodiments, the diagnostic system is based on anatomic divisions, e.g. organ systems. This is meant to mimic traditional frameworks used in physician reasoning in which an organ-based approach can be employed for formulation of a differential diagnosis.

In some embodiments, a logistic regression classifier is used to allow straightforward identification of relevant clinical features and ease of establishing transparency for the diagnostic classification.

In some embodiments, the first level of the diagnostic system categorizes the EHR notes into broad organ systems such as: respiratory, gastrointestinal, neuropsychiatric, genitourinary, and generalized systemic conditions. In some embodiments, this is the only level of separation in the diagnostic hierarchy. In some embodiments, this was the first level of separation in the diagnostic hierarchy. In some embodiments, within at least one of the organ systems in the first level, further sub-classifications and hierarchical layers are made. In some embodiments, the organ systems used in the diagnostic hierarchy comprise at least one of integumentary system, muscular system, skeletal system, nervous system, circulatory system, lymphatic system, respiratory system, endocrine system, urinary/excretory system, reproductive system, and digestive system. In some embodiments, the diagnostic system comprises multiple levels of categorization such as a first level, a second level, a third level, a fourth level, and/or a fifth level. In some embodiments, the diagnostic system comprises at least two levels, at least three levels, at least four levels, or at least five levels of categorization. For example, in some embodiments, the respiratory system is further divided into upper respiratory conditions and lower respiratory conditions. Next, the conditions are further separated into more specific anatomic divisions (e.g. laryngitis, tracheitis, bronchitis, pneumonia). FIG. 3 illustrates an embodiment of hierarchical classification of pediatric diseases. As shown in FIG. 3, general pediatric diseases are classified in a first level into respiratory diseases, genitourinary diseases, gastrointestinal diseases, systemic generalized diseases, and neuropsychiatric diseases. In some embodiments, respiratory diseases are further classified into upper or lower respiratory diseases. In some embodiments, upper respiratory diseases are further classified into acute upper respiratory infection, sinusitis, or acute laryngitis. In some embodiments, sinusitis is further classified into acute sinusitis or acute recurrent sinusitis. In some embodiments, lower respiratory disease is further classified into bronchitis, pneumonia, asthma, or acute tracheitis. In some embodiments, bronchitis is further classified into acute bronchitis, bronchiolitis, or acute bronchitis due to mycoplasma pneumonia. In some embodiments, pneumonia is further classified into bacterial pneumonia or mycoplasma infection. In some embodiments, bacterial pneumonia is further classified into bronchopneumonia or bacterial pneumonia (elsewhere). In some embodiments, asthma is further classified into asthma (uncomplicated), cough variant asthma, or asthma with acute exacerbation. In some embodiments, gastrointestinal disease is further classified into diarrhea, mouth-related diseases, or acute pharyngitis. In some embodiments, systemic generalized disease is further classified into hand, foot & mouth disease, varicella (without complications), influenza, infectious mononucleosis, sepsis, or exanthema subitum. In some embodiments, neuropsychiatric disease is further classified into tic disorder, attention-deficit hyperactivity disorder, bacterial meningitis, encephalitis, or convulsions.

In some embodiments, the performance of the classifier is evaluated at each level of the diagnostic hierarchy. Accordingly, in some embodiments, the system is designed to evaluate the extracted features of each patient record and categorize the set of features into finer levels of diagnostic specificity along the levels of the decision tree, similar to how a human physician might evaluate a patient's features to achieve a diagnosis based on the same clinical data incorporated into the information model. In some embodiments, encounters labeled by physicians as having a primary diagnosis of “fever” or “cough” are eliminated, as these represented symptoms rather than specific disease entities.

In some embodiments, across all levels of the diagnostic hierarchy, this diagnostic system achieved a high level of accuracy between the predicted primary diagnoses based on the extracted clinical features by the NLP information model and the initial diagnoses designated by the examining physician (see Table 3 in Example 1). For the first level where the diagnostic system classified the patient's diagnosis into a broad organ system, the median accuracy was 0.90, ranging from 0.85 for gastrointestinal diseases to 0.98 for neuropsychiatric disorders (see Table 3a of Example 1). Even at deeper levels of diagnostic specification, the system retained a strong level of performance. To illustrate, within the respiratory system, the next division in the diagnostic hierarchy was between upper respiratory and lower respiratory conditions. The system achieved an accuracy of 0.89 of upper respiratory conditions and 0.87 of lower respiratory conditions between predicted diagnoses and initial diagnoses (Table 3b). When dividing the upper respiratory subsystem into more specific categories, the median accuracy was 0.92 (range: 0.86 for acute laryngitis to 0.96 for sinusitis, Table 3c). Acute upper respiratory infection was the single most common diagnosis among the cohort, and the model was able to accurately predict the diagnosis in 95% of the encounters (Table 3c). Within the respiratory system, asthma was categorized separately as its own subcategory, and the accuracy ranged from 0.83 for cough variant asthma to 0.97 for unspecified asthma with acute exacerbation (Table 3d).

In some embodiments, the diagnostic model described herein is assessed according to one or more performance metrics. In some embodiments, the model has an accuracy of at least 70%, at least 75%, at least 80%, at least 85%, at least 90%, at least 91%, at least 92%, at least 93%, at least 94%, at least 95%, at least 96%, at least 97%, at least 98%, or at least 99% for at least 200 independent samples. In some embodiments, the model produces a sensitivity of at least 70%, at least 75%, at least 80%, at least 85%, at least 90%, at least 91%, at least 92%, at least 93%, at least 94%, at least 95%, at least 96%, at least 97%, at least 98%, or at least 99% for at least 200 independent samples. In some embodiments, the model produces a specificity of at least 70%, at least 75%, at least 80%, at least 85%, at least 90%, at least 91%, at least 92%, at least 93%, at least 94%, at least 95%, at least 96%, at least 97%, at least 98%, or at least 99% for at least 200 independent samples. In some embodiments, the model produces a positive predictive value of at least 70%, at least 75%, at least 80%, at least 85%, at least 90%, at least 91%, at least 92%, at least 93%, at least 94%, at least 95%, at least 96%, at least 97%, at least 98%, or at least 99% for at least 200 independent samples. In some embodiments, the model produces a negative predictive value of at least 70%, at least 75%, at least 80%, at least 85%, at least 90%, at least 91%, at least 92%, at least 93%, at least 94%, at least 95%, at least 96%, at least 97%, at least 98%, or at least 99% for at least 200 independent samples.

Identification of Common Features Driving Diagnostic Prediction

Disclosed herein are systems and methods for gaining insight into how the diagnostic system utilizes the clinical features extracted by the deep NLP information model and generates a predicted diagnosis. In some embodiments, the key clinical features driving the diagnosis prediction are identified. For each feature, the category of EHR clinical data the feature was derived from (e.g. history of present illness, physical exam, etc.) is determined along with its classification (e.g. binary or free text classification). This ability to review clinical features driving the computer-predicted diagnosis allowed an evaluation as to whether the prediction was based on clinically relevant features. In some embodiments, these features are provided and/or explained to the user or subject (e.g. patient or a healthcare provider diagnosing and/or treating the patient) to build transparency and trust of the diagnosis and diagnostic system.

For instance, taking gastroenteritis as an example, the diagnostic system identified the presence of words such as “abdominal pain” and “vomiting” as key associated clinical features. The binary classifiers were coded such that presence of the feature was denoted as “1” and absence was denoted as “0”. In this case, “vomiting=1” and “abdominal pain=1” were identified as key features for both chief complaint and history of present illness. Under physical exam, “abdominal tenderness=1” and “rash=1” were noted to be associated with this diagnosis. Interestingly, “palpable mass=0” was also associated, meaning that the patients predicted to have gastroenteritis usually did not have a palpable mass, which is consistent with human clinical experience. In addition to binary classifiers, there were also “free text” categories in the schema. The feature of “fever” with a text entry of greater than 39 degrees Celsius also emerged as an associated clinical feature driving the diagnosis for gastroenteritis. Laboratory and imaging features were not identified as strongly driving the prediction of this diagnosis, perhaps reflecting the fact that most cases of gastroenteritis are diagnosed without extensive ancillary testing.

### Diagnostic Platforms, Systems, Devices, and Media

Provided herein, in certain aspects, are platforms, systems, devices, and media for analyzing medical data according to any of the methods of the present disclosure. In some embodiments, the systems and electronic devices are integrated with a program including instructions executable by a processor to carry out analysis of medical data. In some embodiments, the analysis comprises processing medical data for at least one subject with a classifier generated and trained using EHRs. In some embodiments, the analysis is performed locally on the device utilizing local software integrated into the device. In some embodiments, the analysis is performed remotely on the cloud after the medical data is uploaded by the system or device over a network. In some embodiments, the system or device is an existing system or device adapted to interface with a web application operating on the network or cloud for uploading and analyzing medical data such as an EHR (or alternatively, a feature set extracted from the EHR containing the relevant clinical features for disease diagnosis/classification).

In some aspects, disclosed herein is a computer-implemented system configured to carry out cloud-based analysis of medical data such as electronic health records. In some embodiments, the cloud-based analysis is performed on batch uploads of data. In some embodiments, the cloud-based analysis is performed in real-time on individual or small groupings of medical data for one or more subjects. In some embodiments, a batch of medical data comprises medical data for at least 5 subjects, at least 10 subjects, at least 20 subjects, at least 30 subjects, at least 40 subjects, at least 50 subjects, at least 60 subjects, at least 70 subjects, at least 80 subjects, at least 90 subjects, at least 100 subjects, at least 150 subjects, at least 200 subjects, at least 300 subjects, at least 400 subjects, or at least 500 subjects.

In some embodiments, the electronic device comprises a user interface for communicating with and/or receiving instructions from a user or subject, a memory, at least one processor, and non-transitory computer readable media providing instructions executable by the at least one processor for analyzing medical data. In some embodiments, the electronic device comprises a network component for communicating with a network or cloud. The network component is configured to communicate over a network using wired or wireless technology. In some embodiments, the network component communicates over a network using Wi-Fi, Bluetooth, 2G, 3G, 4G, 4G LTE, 5G, WiMAX, WiMAN, or other radiofrequency communication standards and protocols.

In some embodiments, the system or electronic device obtains medical data such as one or more electronic health records. In some embodiments, the electronic health records are merged and/or analyzed collectively. In some embodiments, the electronic device is not configured to carry out analysis of the medical data, instead uploading the data to a network for cloud-based or remote analysis. In some embodiments, the electronic device comprises a web portal application that interfaces with the network or cloud for remote analysis and does not carry out any analysis locally. An advantage of this configuration is that medical data is not stored locally and thus less vulnerable to being hacked or lost. Alternatively or in combination, the electronic device is configured to carry out analysis of the medical data locally. An advantage of this configuration is the ability to perform analysis in locations lacking network access or coverage (e.g. in certain remote locations lacking internet coverage). In some embodiments, the electronic device is configured to carry out analysis of the medical data locally when network access is not available as a backup function such as in case of an internet outage or temporary network failure. In some embodiments, the medical data is uploaded for storage on the cloud regardless of where the analysis is carried out. For example, in certain instances, the medical data is temporarily stored on the electronic device for analysis, and subsequently uploaded on the cloud and/or deleted from the electronic device's local memory.

In some embodiments, the electronic device comprises a display for providing the results of the analysis such as a diagnosis or prediction (of the presence and/or progression of a disease or disorder), a treatment recommendation, treatment options, healthcare provider information (e.g. nearby providers that can provide the recommended treatment and/or confirm the diagnosis), or a combination thereof. In some embodiments, the diagnosis or prediction is generated from analysis of current medical data (e.g. most recent medical data or EHR entered for analysis) in comparison to historical medical data (e.g. medical data or EHR from previous medical visits) for the same subject to determine the progression of a disease or disorder. In some embodiments, the medical data such as electronic health records are time-stamped. In some embodiments, electronic health records are stored as data, which optionally includes meta-data such as a timestamp, location, user info, or other information. In some embodiments, the electronic device comprises a portal providing tools for a user to input information such as name, address, email, phone number, and/or other identifying information. In some embodiments, the portal provides tools for inputting or uploading medical information (e.g. EHRs, blood pressure, temperature, symptoms, etc.). In some embodiments, the portal provides the user with the option to receive the results of the analysis by email, messaging (e.g. SMS, text message), physical printout (e.g. a printed report), social media, by phone (e.g. an automated phone message or a consultation by a healthcare provider or adviser), or a combination thereof. In some embodiments, the portal is displayed on a digital screen of the electronic device. In some embodiments, the electronic device comprises an analog interface. In some embodiments, the electronic device comprises a digital interface such as a touchscreen.

In some embodiments, disclosed herein is an online diagnosis, triage, and/or referral AI system. In some embodiments, the system utilizes keywords extracted from an EHR or other data. In some embodiments, the system generates a diagnosis based on analysis of the keywords. In some embodiments, the diagnosis is used to triage a patient relative to a plurality of patients. In some embodiments, the diagnosis is used to refer a patient to a healthcare provider.

### Digital Processing Device

In some embodiments, the platforms, media, methods and applications described herein include or utilize a digital processing device, a processor, or use of the same. In some embodiments, a digital processing device is configured to perform any of the methods described herein such as generating a natural language processing information extraction model and/or utilizing said model to analyze medical data such as EHRs. In further embodiments, the digital processing device includes one or more processors or hardware central processing units (CPU) that carry out the device's functions. In still further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device. In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In some embodiments, the non-volatile memory comprises magnetoresistive random-access memory (MRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a subject. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In some embodiments, the display is E-paper or E ink. In other embodiments, the display is a video projector. In still further embodiments, the display is a combination of devices such as those disclosed herein.

In some embodiments, the digital processing device includes an input device to receive information from a subject. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

### Non-Transitory Computer Readable Storage Medium

In some embodiments, the platforms, media, methods and applications described herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

### Computer Program

In some embodiments, the platforms, media, methods and applications described herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, a computer program includes a mobile application provided to a mobile digital processing device such as a smartphone. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g. not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, media, methods and applications described herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of barcode, route, parcel, subject, or network information. In various embodiments, suitable databases include, by way of non-limiting examples, relational databases, non-relational databases, object oriented databases, object databases, entity-relationship model databases, associative databases, and XML databases. In some embodiments, a database is internet-based. In further embodiments, a database is web-based. In still further embodiments, a database is cloud computing-based. In other embodiments, a database is based on one or more local computer storage devices.

### DETAILED FIGURE DESCRIPTIONS

FIG. 1 shows the results of unsupervised clustering of pediatric diseases. The diagnostic system described herein analyzed electronic health records in the absence of a defined classification system. This grouping structure reflects the detection of trends in clinical features by the deep-learning based model without pre-defined labeling or human input. The clustered blocks are marked with the boxes with grey lines.

FIG. 2 shows an embodiment of a workflow diagram depicting the process of data extraction from electronic medical records, followed by deep learning-based natural language processing (NLP) analysis of these encounters, which were then processed with a disease classifier to predict a clinical diagnosis for each encounter.

FIG. 3 shows an example of a hierarchy of the diagnostic framework in a large pediatric cohort. A logistic regression classifier was used to establish a diagnostic system based on anatomic divisions. An organ-based approach was used, wherein diagnoses were first separated into broad organ systems, and then subsequently divided into organ subsystems and/or into more specific diagnosis groups.

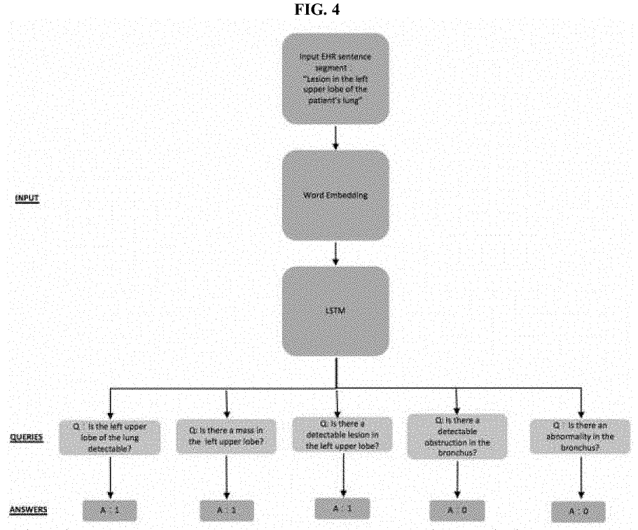

FIG. 4 shows an example of a design of the natural language processing (NLP) information extraction model. Segmented sentences from the raw text of the electronic health record were embedded using word2vec. The LSTM model then output the structured records in query answer format. In this particular example, a sample EHR sentence segment is used as input (“Lesion in the left upper lobe of the patient's lung”). Next, word embedding is performed, followed by sentence classification using Long Short Term Memory (LSTM) architecture. Finally, the input is evaluated against a set of queries and their corresponding answers. Specifically, the queries shown in FIG. 4 include in order from left to right: “Q: Is the upper left lobe of the lung detectable?”/“A: 1”; “Q: Is there a mass in the upper left lobe?”/“A: 1”; “Q: Is there a detectable lesion in the upper left lobe?”/“A: 1”; “Q: Is there a detectable obstruction in the bronchus”/“A: 0”; “Q: Is there an abnormality in the bronchus”/“A: 0”.

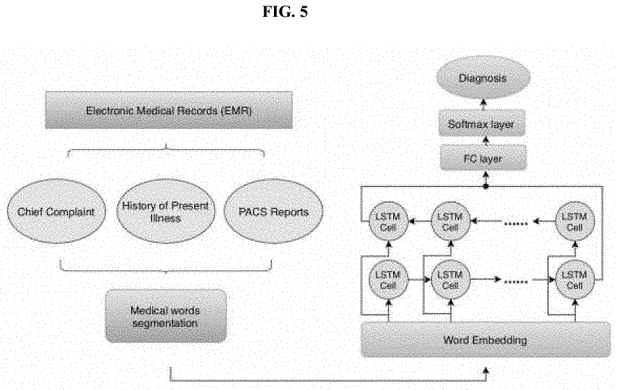

FIG. 5 shows a workflow diagram that depicts an embodiment of the hybrid natural language processing and machine learning AI-based system. A comprehensive medical dictionary and open-source Chinese language segmentation software was applied to EHR data as a means to extract clinically relevant text. This information was fed through a NLP analysis and then processed with a disease classifier to predict a diagnosis for each encounter.

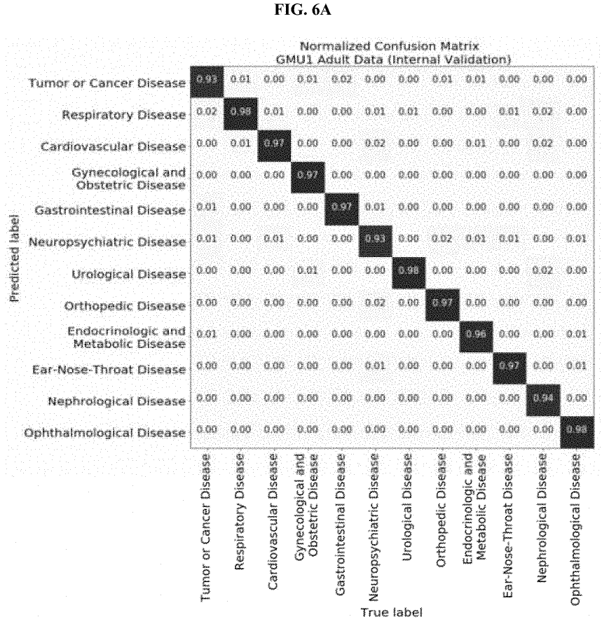

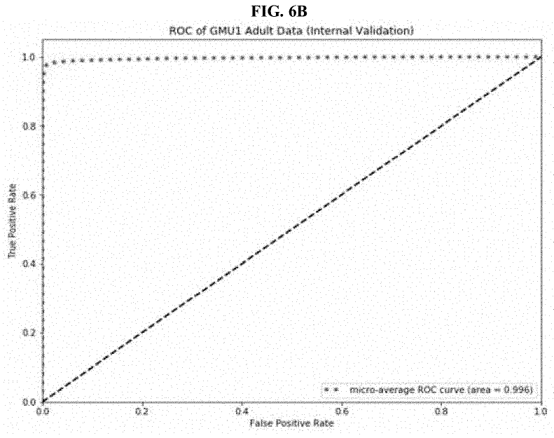

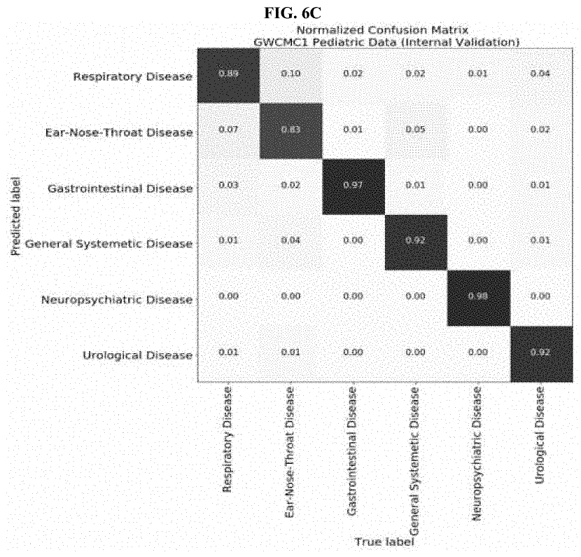

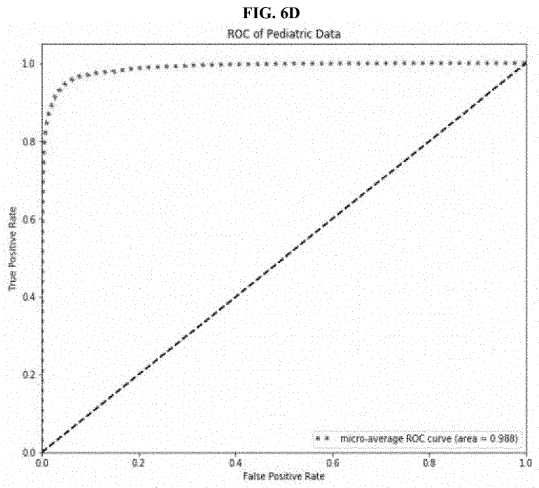

FIGS. 6A-6D shows the diagnostic efficiencies and model performance for GMU1 adult data and GWCMC1 pediatric data. FIG. 6A shows a convolutional table showing diagnostic efficiencies across adult populations. FIG. 6B shows an ROC-AUC curve for model performance across adult populations. FIG. 6C shows a convolutional table showing diagnostic efficiencies across pediatric populations. FIG. 6D shows an ROC-AUC curve for model performance across pediatric populations.

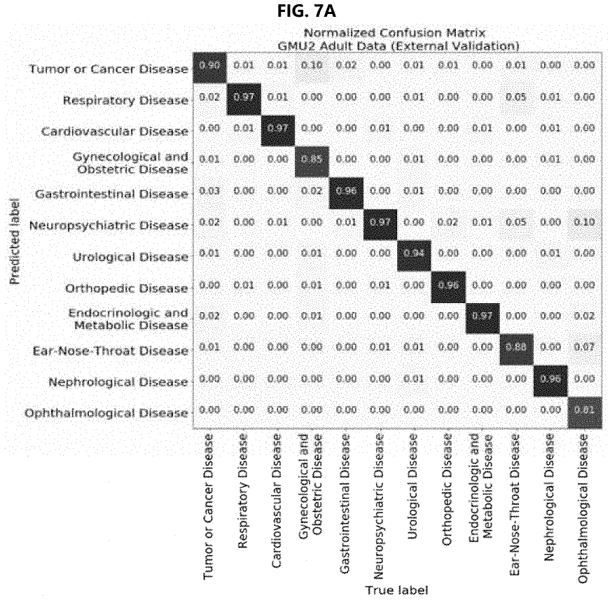

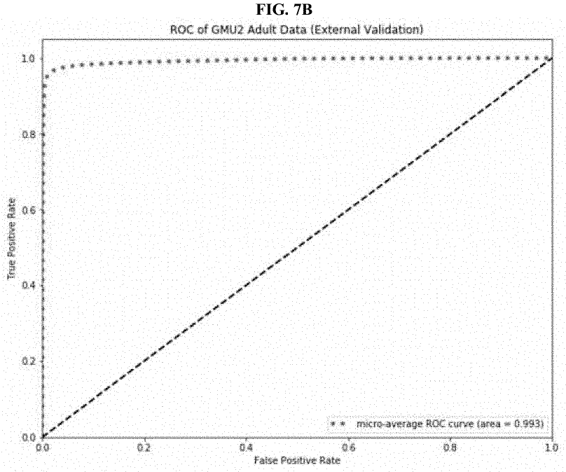

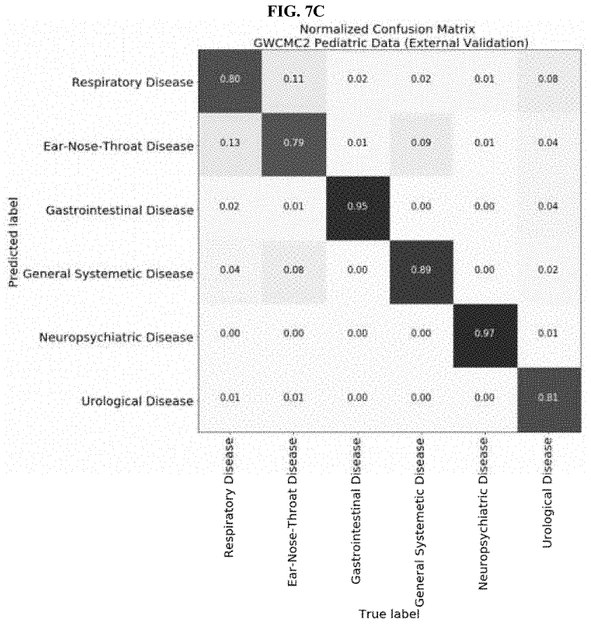

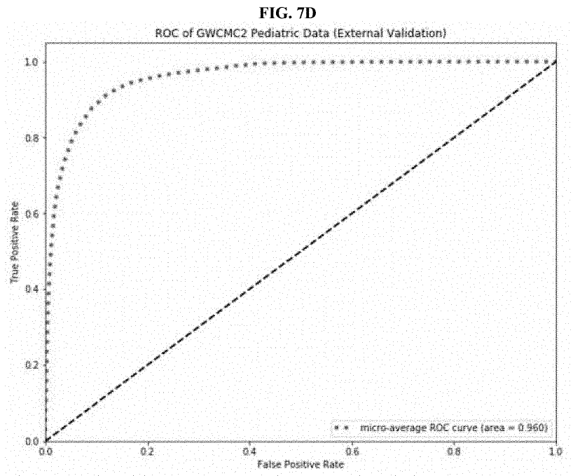

FIGS. 7A-7D shows the diagnostic efficiencies and model performance for GMU2 adult data and GWCMC2 pediatric data. FIG. 7A shows a convolutional table showing diagnostic efficiencies across adult populations. FIG. 7B shows an ROC-AUC curve for model performance across adult populations. FIG. 7C shows a convolutional table showing diagnostic efficiencies across pediatric populations. FIG. 7D shows an ROC-AUC curve for model performance across pediatric populations.

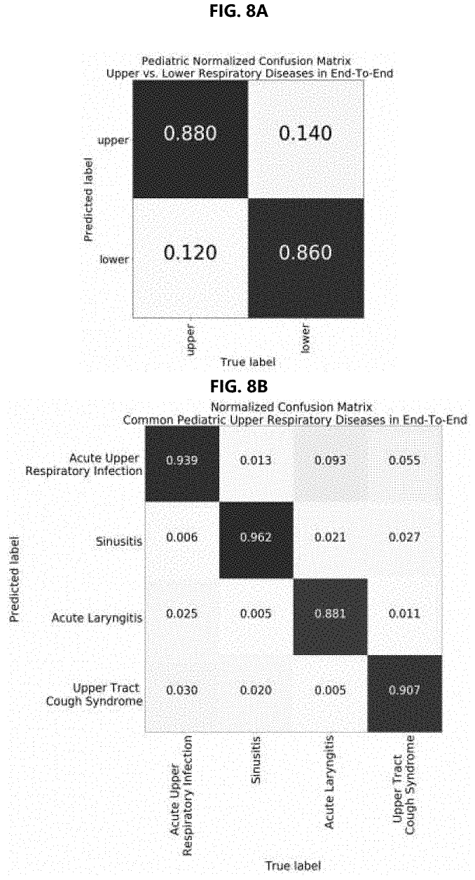

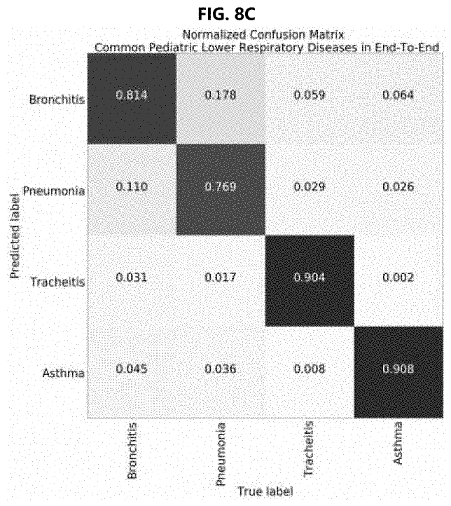

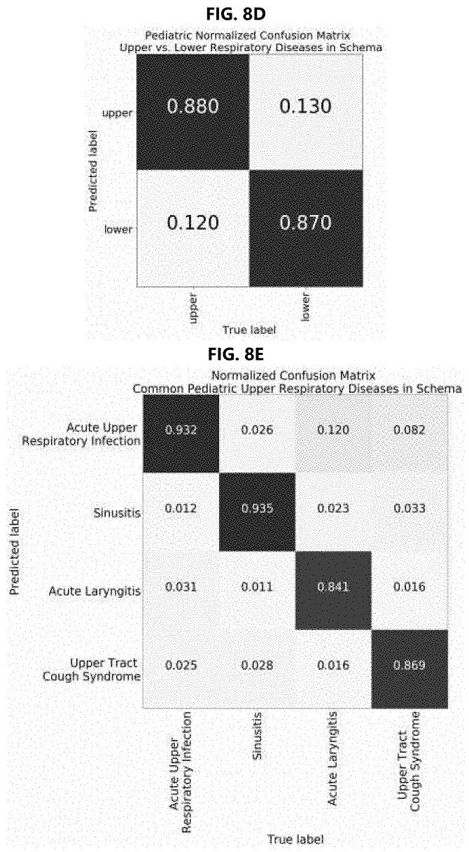

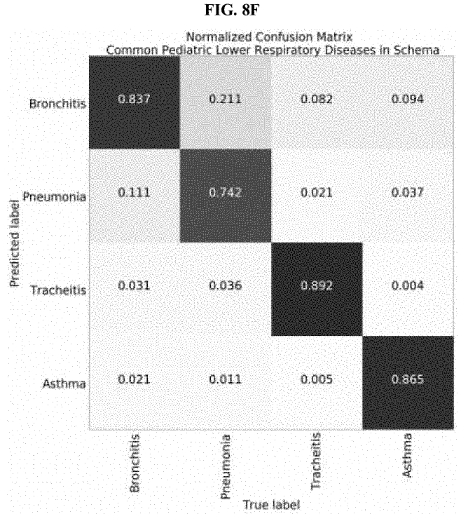

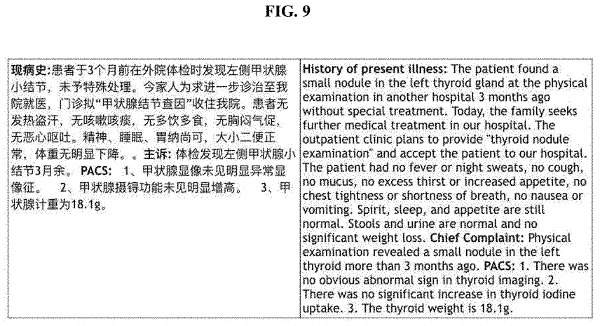

FIGS. 8A-8F shows Comparison of Hierarchical Diagnosis Approach (right) versus end-to-end approach in pediatric respiratory diseases (left). FIGS. 8A-8C shows an end-to-end approach. FIG. 8A depicts a confusion table showing diagnostic efficiencies between upper and lower respiratory systems in pediatric patients. FIG. 8B depicts a confusion table showing diagnostic efficiencies in top four upper-respiratory diseases. FIG. 8C shows a confusion table showing diagnostic efficiencies in top six lower-respiratory diseases. FIGS. 8D-8F show a hierarchical diagnostic approach. FIG. 8D depicts a confusion table showing diagnostic efficiencies for upper and lower respiratory systems in pediatric patients. FIG. 8E depicts a confusion table showing diagnostic efficiencies in top four upper-respiratory diseases. FIG. 8F depicts a confusion table showing diagnostic efficiencies in top six lower-respiratory diseases.

FIG. 9 shows an example of free-text document record of an endocrinological and metabolic disease case that can be used in segmentation.

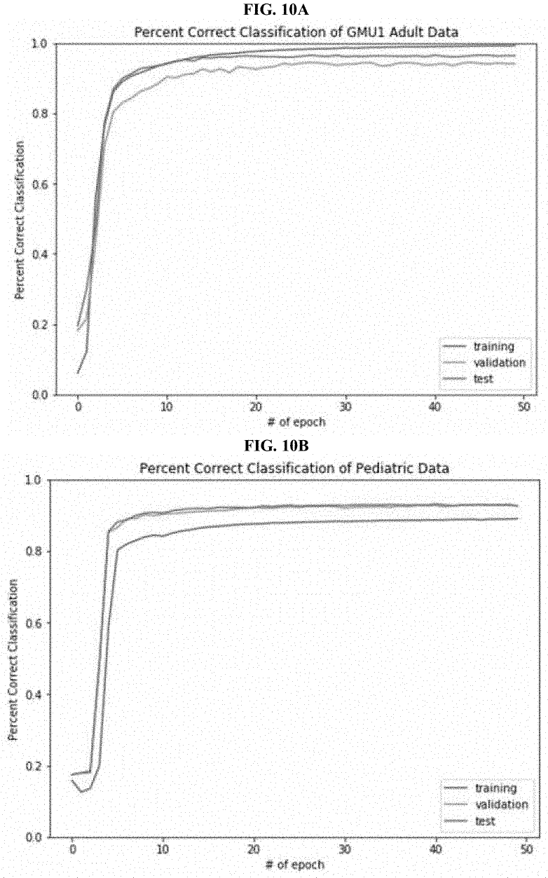

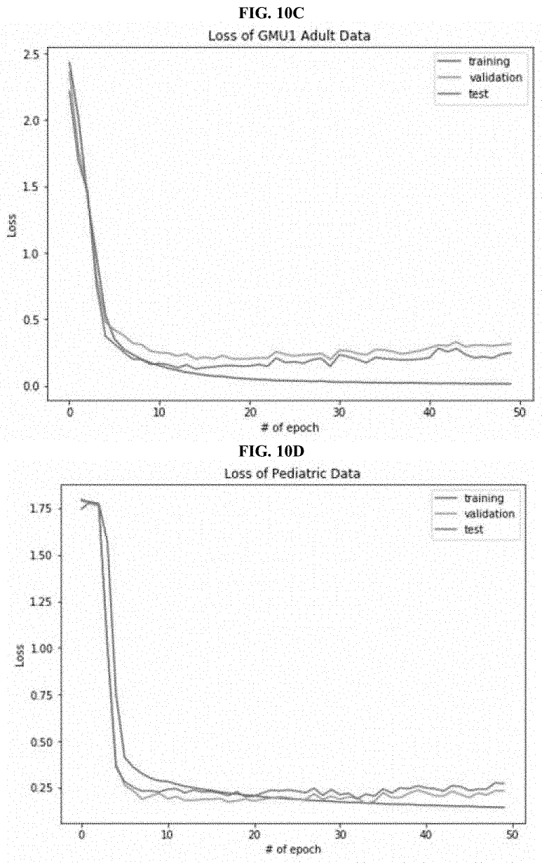

FIG. 10A-FIG. 10D show model performance over time with percent classification and loss over number of epochs in adult and pediatric internal validations.

### NUMBERED EMBODIMENTS

The following embodiments recite nonlimiting permutations of combinations of features disclosed herein. Other permutations of combinations of features are also contemplated. A method for providing a medical diagnosis, comprising: obtaining medical data; using a natural language processing (NLP) information extraction model to extract and annotate clinical features from the medical data; and analyzing at least one of the clinical features with a disease prediction classifier to generate a classification of a disease or disorder, the classification having a sensitivity of at least 80%. The method of embodiment 1, wherein the NLP information extraction model comprises a deep learning procedure. The method of embodiment 1 or 2, wherein the NLP information extraction model utilizes a standard lexicon comprising keywords representative of assertion classes. The method of any one of embodiments 1-3, wherein the NLP information extraction model utilizes a plurality of schema, each schema comprising a feature name, anatomical location, and value. The method of embodiment 4, wherein the plurality of schema comprises at least one of history of present illness, physical examination, laboratory test, radiology report, and chief complaint. The method any one of embodiments 1-5, further comprising tokenizing the medical data for processing by the NLP information extraction model. The method of any one of embodiments 1-6, wherein the medical data comprises an electronic health record (EHR). The method of any one of embodiments 1-7, wherein the classification has a specificity of at least 80%. The method of any one of embodiments 1-8, wherein the classification has an F1 score of at least 80%. The method of any of embodiments 1-9, wherein the clinical features are extracted in a structured format comprising data in query-answer pairs. The method of any of embodiments 1-10, wherein the disease prediction classifier comprises a logistic regression classifier. The method of any one of embodiments 1-11, wherein the disease prediction classifier comprises a decision tree. The method of any one of embodiments 1-12, wherein the classification differentiates between a serious and a non-serious condition. The method of any one of embodiments 1-13, wherein the classification comprises at least two levels of categorization. The method of any one of embodiments 1-14, wherein the classification comprises a first level category indicative of an organ system. The method of embodiments 15, wherein the classification comprises a second level indicative of a subcategory of the organ system. The method of any one of embodiments 1-16, wherein the classification comprises a diagnostic hierarchy that categorizes the disease or disorder into a series of narrower categories. The method of embodiment 16, wherein the classification comprises a categorization selected from the group consisting of respiratory diseases, genitourinary diseases, gastrointestinal diseases, neuropsychiatric diseases, and systemic generalized diseases. The method of embodiment 18, wherein the classification further comprises a subcategorization of respiratory diseases into upper respiratory diseases and lower respiratory diseases. The method of embodiment 19, wherein the classification further comprises a subcategorization of upper respiratory disease into acute upper respiratory disease, sinusitis, or acute laryngitis. The method of embodiment 19, wherein the classification further comprises a subcategorization of lower respiratory disease into bronchitis, pneumonia, asthma, or acute tracheitis. The method of embodiment 18, wherein the classification further comprises a subcategorization of gastrointestinal diseases into diarrhea, mouth-related diseases, or acute pharyngitis. The method of embodiment 18, wherein the classification further comprises a subcategorization of neuropsychiatric diseases into tic disorder, attention-deficit hyperactivity disorder, bacterial meningitis, encephalitis, or convulsions. The method of embodiment 18, wherein the classification further comprises a subcategorization of systemic generalized diseases into hand, foot and mouth disease, varicella without complication, influenza, infectious mononucleosis, sepsis, or exanthema subitum. The method of any one of embodiments 1-24, further comprising making a medical treatment recommendation based on the classification. The method of any one of embodiments 1-25, wherein the disease prediction classifier is trained using end-to-end deep learning. A non-transitory computer-readable medium comprising machine-executable code that, upon execution by one or more computer processors, implements a method for providing a classification of a disease or disorder, the method comprising: obtaining medical data; using a natural language processing (NLP) information extraction model to extract and annotate clinical features from the medical data; and analyzing at least one of the clinical features with a disease prediction classifier to generate the classification of a disease or disorder, the classification having a sensitivity of at least 80%. The media of embodiment 27, wherein the NLP information extraction model comprises a deep learning procedure. The media of embodiment 27 or 28, wherein the NLP information extraction model utilizes a standard lexicon comprising keywords representative of assertion classes. The media of any one of embodiments 27-29, wherein the NLP information extraction model utilizes a plurality of schema, each schema comprising a feature name, anatomical location, and value. The media of embodiment 30, wherein the plurality of schema comprises at least one of history of present illness, physical examination, laboratory test, radiology report, and chief complaint. The media of any one of embodiments 27-31, wherein the method further comprises tokenizing the medical data for processing by the NLP information extraction model. The media of any one of embodiments 27-32, wherein the medical data comprises an electronic health record (EHR). The media of any one of embodiments 27-33, wherein the classification has a specificity of at least 80%. The media of any one of embodiments 27-34, wherein the classification has an F1 score of at least 80%. The media of any of embodiments 27-35, wherein the clinical features are extracted in a structured format comprising data in query-answer pairs. The media of any of embodiments 27-36, wherein the disease prediction classifier comprises a logistic regression classifier. The media of any one of embodiments 27-37, wherein the disease prediction classifier comprises a decision tree. The media of any one of embodiments 27-38, wherein the classification differentiates between a serious and a non-serious condition. The media of any one of embodiments 27-39, wherein the classification comprises at least two levels of categorization. The media of any one of embodiments 27-40, wherein the classification comprises a first level category indicative of an organ system. The media of embodiments 41, wherein the classification comprises a second level indicative of a subcategory of the organ system. The media of any one of embodiments 27-42, wherein the classification comprises a diagnostic hierarchy that categorizes the disease or disorder into a series of narrower categories. The media of embodiment 43, wherein the classification comprises a categorization selected from the group consisting of respiratory diseases, genitourinary diseases, gastrointestinal diseases, neuropsychiatric diseases, and systemic generalized diseases. The media of embodiment 44, wherein the classification further comprises a subcategorization of respiratory diseases into upper respiratory diseases and lower respiratory diseases. The media of embodiment 45, wherein the classification further comprises a subcategorization of upper respiratory disease into acute upper respiratory disease, sinusitis, or acute laryngitis. The media of embodiment 45, wherein the classification further comprises a subcategorization of lower respiratory disease into bronchitis, pneumonia, asthma, or acute tracheitis. The media of embodiment 44, wherein the classification further comprises a subcategorization of gastrointestinal diseases into diarrhea, mouth-related diseases, or acute pharyngitis. The media of embodiment 44, wherein the classification further comprises a subcategorization of neuropsychiatric diseases into tic disorder, attention-deficit hyperactivity disorder, bacterial meningitis, encephalitis, or convulsions. The media of embodiment 44, wherein the classification further comprises a subcategorization of systemic generalized diseases into hand, foot and mouth disease, varicella without complication, influenza, infectious mononucleosis, sepsis, or exanthema subitum. The media of any one of embodiments 27-50, further comprising making a medical treatment recommendation based on the classification. The media of any one of embodiments 27-51, wherein the disease prediction classifier is trained using end-to-end deep learning. A computer-implemented system comprising: a digital processing device comprising: at least one processor, an operating system configured to perform executable instructions, a memory, and a computer program including instructions executable by the digital processing device to create an application for providing a medical diagnosis, the application comprising: a software module obtaining medical data; a software module using a natural language processing (NLP) information extraction model to extract and annotate clinical features from the medical data; and a software module analyzing at least one of the clinical features with a disease prediction classifier to generate the classification of a disease or disorder, the classification having a sensitivity of at least 80%. The system of embodiment 53, wherein the NLP information extraction model comprises a deep learning procedure. The system of embodiment 53 or 54, wherein the NLP information extraction model utilizes a standard lexicon comprising keywords representative of assertion classes. The system of any one of embodiments 53-55, wherein the NLP information extraction model utilizes a plurality of schema, each schema comprising a feature name, anatomical location, and value. The system of embodiment 56, wherein the plurality of schema comprises at least one of history of present illness, physical examination, laboratory test, radiology report, and chief complaint. The system of any one of embodiments 53-57, further comprising a software module tokenizing the medical data for processing by the NLP information extraction model. The system of any one of embodiments 53-58, wherein the medical data comprises an electronic health record (EHR). The system of any one of embodiments 53-59, wherein the classification has a specificity of at least 80%. The system of any one of embodiments 53-60, wherein the classification has an F1 score of at least 80%. The system of any of embodiments 53-61, wherein the clinical features are extracted in a structured format comprising data in query-answer pairs. The system of any of embodiments 53-62, wherein the disease prediction classifier comprises a logistic regression classifier. The system of any one of embodiments 53-63, wherein the disease prediction classifier comprises a decision tree. The system of any one of embodiments 53-64, wherein the classification differentiates between a serious and a non-serious condition. The system of any one of embodiments 53-65, wherein the classification comprises at least two levels of categorization. The system of any one of embodiments 53-66, wherein the classification comprises a first level category indicative of an organ system. The system of embodiments 67, wherein the classification comprises a second level indicative of a subcategory of the organ system. The system of any one of embodiments 53-68, wherein the classification comprises a diagnostic hierarchy that categorizes the disease or disorder into a series of narrower categories. The system of embodiment 69, wherein the classification comprises a categorization selected from the group consisting of respiratory diseases, genitourinary diseases, gastrointestinal diseases, neuropsychiatric diseases, and systemic generalized diseases. The system of embodiment 70, wherein the classification further comprises a subcategorization of respiratory diseases into upper respiratory diseases and lower respiratory diseases. The system of embodiment 71, wherein the classification further comprises a subcategorization of upper respiratory disease into acute upper respiratory disease, sinusitis, or acute laryngitis. The system of embodiment 71, wherein the classification further comprises a subcategorization of lower respiratory disease into bronchitis, pneumonia, asthma, or acute tracheitis. The system of embodiment 70, wherein the classification further comprises a subcategorization of gastrointestinal diseases into diarrhea, mouth-related diseases, or acute pharyngitis. The system of embodiment 70, wherein the classification further comprises a subcategorization of neuropsychiatric diseases into tic disorder, attention-deficit hyperactivity disorder, bacterial meningitis, encephalitis, or convulsions. The system of embodiment 70, wherein the classification further comprises a subcategorization of systemic generalized diseases into hand, foot and mouth disease, varicella without complication, influenza, infectious mononucleosis, sepsis, or exanthema subitum. The system of any one of embodiments 53-76, further comprising making a medical treatment recommendation based on the classification. The system of any one of embodiments 53-77, wherein the disease prediction classifier is trained using end-to-end deep learning. A digital processing device comprising: at least one processor, an operating system configured to perform executable instructions, a memory, and a computer program including instructions executable by the digital processing device to create an application for providing a medical diagnosis, the application comprising: a software module obtaining medical data; a software module using a natural language processing (NLP) information extraction model to extract and annotate clinical features from the medical data; and a software module analyzing at least one of the clinical features with a disease prediction classifier to generate the classification of a disease or disorder, the classification having a sensitivity of at least 80%. The device of embodiment 79, wherein the NLP information extraction model comprises a deep learning procedure. The device of embodiment 79 or 80, wherein the NLP information extraction model utilizes a standard lexicon comprising keywords representative of assertion classes. The device of any one of embodiments 79-81, wherein the NLP information extraction model utilizes a plurality of schema, each schema comprising a feature name, anatomical location, and value. The device of embodiment 82, wherein the plurality of schema comprises at least one of history of present illness, physical examination, laboratory test, radiology report, and chief complaint. The device of any one of embodiments 79-83, further comprising a software module tokenizing the medical data for processing by the NLP information extraction model. The device of any one of embodiments 79-84, wherein the medical data comprises an electronic health record (EHR). The device of any one of embodiments 79-85, wherein the classification has a specificity of at least 80%. The device of any one of embodiments 79-86, wherein the classification has an F1 score of at least 80%. The device of any of embodiments 79-87, wherein the clinical features are extracted in a structured format comprising data in query-answer pairs. The device of any of embodiments 79-88, wherein the disease prediction classifier comprises a logistic regression classifier. The device of any one of embodiments 79-89, wherein the disease prediction classifier comprises a decision tree. The device of any one of embodiments 79-90, wherein the classification differentiates between a serious and a non-serious condition. The device of any one of embodiments 79-91, wherein the classification comprises at least two levels of categorization. The device of any one of embodiments 79-92, wherein the classification comprises a first level category indicative of an organ system. The device of embodiments 93, wherein the classification comprises a second level indicative of a subcategory of the organ system. The device of any one of embodiments 79-94, wherein the classification comprises a diagnostic hierarchy that categorizes the disease or disorder into a series of narrower categories. The device of embodiment 95, wherein the classification comprises a categorization selected from the group consisting of respiratory diseases, genitourinary diseases, gastrointestinal diseases, neuropsychiatric diseases, and systemic generalized diseases. The device of embodiment 96, wherein the classification further comprises a subcategorization of respiratory diseases into upper respiratory diseases and lower respiratory diseases. The device of embodiment 97, wherein the classification further comprises a subcategorization of upper respiratory disease into acute upper respiratory disease, sinusitis, or acute laryngitis. The device of embodiment 97, wherein the classification further comprises a subcategorization of lower respiratory disease into bronchitis, pneumonia, asthma, or acute tracheitis. The device of embodiment 96, wherein the classification further comprises a subcategorization of gastrointestinal diseases into diarrhea, mouth-related diseases, or acute pharyngitis. The device of embodiment 96, wherein the classification further comprises a subcategorization of neuropsychiatric diseases into tic disorder, attention-deficit hyperactivity disorder, bacterial meningitis, encephalitis, or convulsions. The device of embodiment 96, wherein the classification further comprises a subcategorization of systemic generalized diseases into hand, foot and mouth disease, varicella without complication, influenza, infectious mononucleosis, sepsis, or exanthema subitum. The device of any one of embodiments 79-102, further comprising making a medical treatment recommendation based on the classification. The device of any one of embodiments 79-103, wherein the disease prediction classifier is trained using end-to-end deep learning. A computer-implemented method for generating a disease prediction classifier for providing a medical diagnosis, comprising: providing a lexicon constructed based on medical texts, wherein the lexicon comprises keywords relating to clinical information; obtaining medical data comprising electronic health records (EHRs); extracting clinical features from the medical data using an NLP information extraction model; mapping the clinical features to hypothetical clinical queries to generate question-answer pairs; and training the NLP classifier using the question-answer pairs, wherein the NLP classifier is configured to generate classifications having a sensitivity of at least 80% when tested against an independent dataset of at least 100 EHRs. The method of embodiment 105, wherein the NLP information extraction model comprises a deep learning procedure. The method of embodiment 105 or 106, wherein the NLP information extraction model utilizes a standard lexicon comprising keywords representative of assertion classes. The method of any one of embodiments 105-107, wherein the NLP information extraction model utilizes a plurality of schema, each schema comprising a feature name, anatomical location, and value. The method of embodiment 108, wherein the plurality of schema comprises at least one of history of present illness, physical examination, laboratory test, radiology report, and chief complaint. The method any one of embodiments 105-109, further comprising tokenizing the medical data for processing by the NLP information extraction model. The method of any one of embodiments 105-110, wherein the medical data comprises an electronic health record (EHR). The method of any one of embodiments 105-111, wherein the classification has a specificity of at least 80%. The method of any one of embodiments 105-112, wherein the classification has an F1 score of at least 80%. The method of any of embodiments 105-113, wherein the clinical features are extracted in a structured format comprising data in query-answer pairs. The method of any of embodiments 105-114, wherein the disease prediction classifier comprises a logistic regression classifier. The method of any one of embodiments 105-115, wherein the disease prediction classifier comprises a decision tree. The method of any one of embodiments 105-116, wherein the classification differentiates between a serious and a non-serious condition. The method of any one of embodiments 105-117, wherein the classification comprises at least two levels of categorization. The method of any one of embodiments 105-118, wherein the classification comprises a first level category indicative of an organ system. The method of embodiments 119, wherein the classification comprises a second level indicative of a subcategory of the organ system. The method of any one of embodiments 105-120, wherein the classification comprises a diagnostic hierarchy that categorizes the disease or disorder into a series of narrower categories. The method of embodiment 120, wherein the classification comprises a categorization selected from the group consisting of respiratory diseases, genitourinary diseases, gastrointestinal diseases, neuropsychiatric diseases, and systemic generalized diseases. The method of embodiment 122, wherein the classification further comprises a subcategorization of respiratory diseases into upper respiratory diseases and lower respiratory diseases. The method of embodiment 123, wherein the classification further comprises a subcategorization of upper respiratory disease into acute upper respiratory disease, sinusitis, or acute laryngitis. The method of embodiment 123, wherein the classification further comprises a subcategorization of lower respiratory disease into bronchitis, pneumonia, asthma, or acute tracheitis. The method of embodiment 122, wherein the classification further comprises a subcategorization of gastrointestinal diseases into diarrhea, mouth-related diseases, or acute pharyngitis. The method of embodiment 122, wherein the classification further comprises a subcategorization of neuropsychiatric diseases into tic disorder, attention-deficit hyperactivity disorder, bacterial meningitis, encephalitis, or convulsions. The method of embodiment 122, wherein the classification further comprises a subcategorization of systemic generalized diseases into hand, foot and mouth disease, varicella without complication, influenza, infectious mononucleosis, sepsis, or exanthema subitum. The method of any one of embodiments 105-128, further comprising making a medical treatment recommendation based on the classification. The method of any one of embodiments 105-129, wherein the disease prediction classifier is trained using end-to-end deep learning. A non-transitory computer-readable medium comprising machine-executable code that, upon execution by one or more computer processors, implements a method for generating a natural language processing (NLP) classifier for providing a classification of a disease or disorder, the method comprising: providing a lexicon constructed based on medical texts, wherein the lexicon comprises keywords relating to clinical information; obtaining medical data comprising electronic health records (EHRs); extracting clinical features from the medical data using an NLP information extraction model; mapping the clinical features to hypothetical clinical queries to generate question-answer pairs; and training the NLP classifier using the question-answer pairs, wherein the NLP classifier is configured to generate classifications having a sensitivity of at least 80% when tested against an independent dataset of at least 100 EHRs. The media of embodiment 131, wherein the NLP information extraction model comprises a deep learning procedure. The media of embodiment 131 or 132, wherein the NLP information extraction model utilizes a standard lexicon comprising keywords representative of assertion classes. The media of any one of embodiments 131-133, wherein the NLP information extraction model utilizes a plurality of schema, each schema comprising a feature name, anatomical location, and value. The media of embodiment 134, wherein the plurality of schema comprises at least one of history of present illness, physical examination, laboratory test, radiology report, and chief complaint. The media of any one of embodiments 131-135, wherein the method further comprises tokenizing the medical data for processing by the NLP information extraction model. The media of any one of embodiments 131-136, wherein the medical data comprises an electronic health record (EHR). The media of any one of embodiments 131-137, wherein the classification has a specificity of at least 80%. The media of any one of embodiments 131-138, wherein the classification has an F1 score of at least 80%. The media of any of embodiments 131-139, wherein the clinical features are extracted in a structured format comprising data in query-answer pairs. The media of any of embodiments 131-140, wherein the disease prediction classifier comprises a logistic regression classifier. The media of any one of embodiments 131-141, wherein the disease prediction classifier comprises a decision tree. The media of any one of embodiments 131-142, wherein the classification differentiates between a serious and a non-serious condition. The media of any one of embodiments 131-143, wherein the classification comprises at least two levels of categorization. The media of any one of embodiments 131-144, wherein the classification comprises a first level category indicative of an organ system. The media of embodiments 145, wherein the classification comprises a second level indicative of a subcategory of the organ system. The media of any one of embodiments 131-146, wherein the classification comprises a diagnostic hierarchy that categorizes the disease or disorder into a series of narrower categories. The media of embodiment 147, wherein the classification comprises a categorization selected from the group consisting of respiratory diseases, genitourinary diseases, gastrointestinal diseases, neuropsychiatric diseases, and systemic generalized diseases. The media of embodiment 148, wherein the classification further comprises a subcategorization of respiratory diseases into upper respiratory diseases and lower respiratory diseases. The media of embodiment 149, wherein the classification further comprises a subcategorization of upper respiratory disease into acute upper respiratory disease, sinusitis, or acute laryngitis. The media of embodiment 149, wherein the classification further comprises a subcategorization of lower respiratory disease into bronchitis, pneumonia, asthma, or acute tracheitis. The media of embodiment 148, wherein the classification further comprises a subcategorization of gastrointestinal diseases into diarrhea, mouth-related diseases, or acute pharyngitis. The media of embodiment 148, wherein the classification further comprises a subcategorization of neuropsychiatric diseases into tic disorder, attention-deficit hyperactivity disorder, bacterial meningitis, encephalitis, or convulsions. The media of embodiment 148, wherein the classification further comprises a subcategorization of systemic generalized diseases into hand, foot and mouth disease, varicella without complication, influenza, infectious mononucleosis, sepsis, or exanthema subitum. The media of any one of embodiments 131-154, further comprising making a medical treatment recommendation based on the classification. The media of any one of embodiments 131-155, wherein the disease prediction classifier is trained using end-to-end deep learning. A computer-implemented system comprising: a digital processing device comprising: at least one processor, an operating system configured to perform executable instructions, a memory, and a computer program including instructions executable by the digital processing device to create an application for generating a natural language processing (NLP) classifier for providing a medical diagnosis, the application comprising: a software module for providing a lexicon constructed based on medical texts, wherein the lexicon comprises keywords relating to clinical information; a software module for obtaining medical data comprising electronic health records (EHRs); a software module for extracting clinical features from the medical data using an NLP information extraction model; a software module for mapping the clinical features to hypothetical clinical queries to generate question-answer pairs; and a software module for training the NLP classifier using the question-answer pairs, wherein the NLP classifier is configured to generate classifications having a sensitivity of at least 80% when tested against an independent dataset of at least 100 EHRs. The system of embodiment 157, wherein the NLP information extraction model comprises a deep learning procedure. The system of embodiment 157 or 158, wherein the NLP information extraction model utilizes a standard lexicon comprising keywords representative of assertion classes. The system of any one of embodiments 157-159, wherein the NLP information extraction model utilizes a plurality of schema, each schema comprising a feature name, anatomical location, and value. The system of embodiment 160, wherein the plurality of schema comprises at least one of history of present illness, physical examination, laboratory test, radiology report, and chief complaint. The system of any one of embodiments 157-161, further comprising a software module tokenizing the medical data for processing by the NLP information extraction model. The system of any one of embodiments 157-162, wherein the medical data comprises an electronic health record (EHR). The system of any one of embodiments 157-163, wherein the classification has a specificity of at least 80%. The system of any one of embodiments 157-164, wherein the classification has an F1 score of at least 80%. The system of any of embodiments 157-165, wherein the clinical features are extracted in a structured format comprising data in query-answer pairs. The system of any of embodiments 157-166, wherein the disease prediction classifier comprises a logistic regression classifier. The system of any one of embodiments 157-167, wherein the disease prediction classifier comprises a decision tree. The system of any one of embodiments 157-168, wherein the classification differentiates between a serious and a non-serious condition. The system of any one of embodiments 157-169, wherein the classification comprises at least two levels of categorization. The system of any one of embodiments 157-170, wherein the classification comprises a first level category indicative of an organ system. The system of embodiments 171, wherein the classification comprises a second level indicative of a subcategory of the organ system. The system of any one of embodiments 157-172, wherein the classification comprises a diagnostic hierarchy that categorizes the disease or disorder into a series of narrower categories. The system of embodiment 173, wherein the classification comprises a categorization selected from the group consisting of respiratory diseases, genitourinary diseases, gastrointestinal diseases, neuropsychiatric diseases, and systemic generalized diseases. The system of embodiment 174, wherein the classification further comprises a subcategorization of respiratory diseases into upper respiratory diseases and lower respiratory diseases. The system of embodiment 175, wherein the classification further comprises a subcategorization of upper respiratory disease into acute upper respiratory disease, sinusitis, or acute laryngitis. The system of embodiment 175, wherein the classification further comprises a subcategorization of lower respiratory disease into bronchitis, pneumonia, asthma, or acute tracheitis. The system of embodiment 174, wherein the classification further comprises a subcategorization of gastrointestinal diseases into diarrhea, mouth-related diseases, or acute pharyngitis. The system of embodiment 174, wherein the classification further comprises a subcategorization of neuropsychiatric diseases into tic disorder, attention-deficit hyperactivity disorder, bacterial meningitis, encephalitis, or convulsions. The system of embodiment 174, wherein the classification further comprises a subcategorization of systemic generalized diseases into hand, foot and mouth disease, varicella without complication, influenza, infectious mononucleosis, sepsis, or exanthema subitum. The system of any one of embodiments 157-180, further comprising making a medical treatment recommendation based on the classification. The system of any one of embodiments 157-181, wherein the disease prediction classifier is trained using end-to-end deep learning. A digital processing device comprising: at least one processor, an operating system configured to perform executable instructions, a memory, and a computer program including instructions executable by the digital processing device to create an application for generating a disease prediction classifier for providing a medical diagnosis, the application comprising: a software module for providing a lexicon constructed based on medical texts, wherein the lexicon comprises keywords relating to clinical information; a software module for obtaining medical data comprising electronic health records (EHRs); a software module for extracting clinical features from the medical data using an NLP information extraction model; a software module for mapping the clinical features to hypothetical clinical queries to generate question-answer pairs; and a software module for training the NLP classifier using the question-answer pairs, wherein the NLP classifier is configured to generate classifications having a sensitivity of at least 80% when tested against an independent dataset of at least 100 EHRs. The device of embodiment 183, wherein the NLP information extraction model comprises a deep learning procedure. The device of embodiment 183 or 183.a), wherein the NLP information extraction model utilizes a standard lexicon comprising keywords representative of assertion classes. The device of any one of embodiments 183-185, wherein the NLP information extraction model utilizes a plurality of schema, each schema comprising a feature name, anatomical location, and value. The device of embodiment 186, wherein the plurality of schema comprises at least one of history of present illness, physical examination, laboratory test, radiology report, and chief complaint. The device of any one of embodiments 183-187, further comprising a software module tokenizing the medical data for processing by the NLP information extraction model. The device of any one of embodiments 183-188, wherein the medical data comprises an electronic health record (EHR). The device of any one of embodiments 183-189, wherein the classification has a specificity of at least 80%. The device of any one of embodiments 183-190, wherein the classification has an F1 score of at least 80%. The device of any of embodiments 183-191, wherein the clinical features are extracted in a structured format comprising data in query-answer pairs. The device of any of embodiments 183-192, wherein the disease prediction classifier comprises a logistic regression classifier. The device of any one of embodiments 183-193, wherein the disease prediction classifier comprises a decision tree. The device of any one of embodiments 183-194, wherein the classification differentiates between a serious and a non-serious condition. The device of any one of embodiments 183-195, wherein the classification comprises at least two levels of categorization. The device of any one of embodiments 183-196, wherein the classification comprises a first level category indicative of an organ system. The device of embodiments 197, wherein the classification comprises a second level indicative of a subcategory of the organ system. The device of any one of embodiments 183-198, wherein the classification comprises a diagnostic hierarchy that categorizes the disease or disorder into a series of narrower categories. The device of embodiment 199, wherein the classification comprises a categorization selected from the group consisting of respiratory diseases, genitourinary diseases, gastrointestinal diseases, neuropsychiatric diseases, and systemic generalized diseases. The device of embodiment 200, wherein the classification further comprises a subcategorization of respiratory diseases into upper respiratory diseases and lower respiratory diseases. The device of embodiment 201, wherein the classification further comprises a subcategorization of upper respiratory disease into acute upper respiratory disease, sinusitis, or acute laryngitis. The device of embodiment 201, wherein the classification further comprises a subcategorization of lower respiratory disease into bronchitis, pneumonia, asthma, or acute tracheitis. The device of embodiment 200, wherein the classification further comprises a subcategorization of gastrointestinal diseases into diarrhea, mouth-related diseases, or acute pharyngitis. The device of embodiment 200, wherein the classification further comprises a subcategorization of neuropsychiatric diseases into tic disorder, attention-deficit hyperactivity disorder, bacterial meningitis, encephalitis, or convulsions. The device of embodiment 200, wherein the classification further comprises a subcategorization of systemic generalized diseases into hand, foot and mouth disease, varicella without complication, influenza, infectious mononucleosis, sepsis, or exanthema subitum. The device of any one of embodiments 183-206, further comprising making a medical treatment recommendation based on the classification. The device of any one of embodiments 183-207, wherein the disease prediction classifier is trained using end-to-end deep learning.

### EXAMPLES

### Example 1

A retrospective study was carried out using electronic health records obtained from electronic health records from Guangzhou Women and Children's Medical Center, a major Chinese academic medical referral center.

Methods

Data Collection

A retrospective study was carried out based on electronic health records obtained from 1,362,559 outpatient patient visits from 567,498 patients from the Guangzhou Women and Children's Medical Center. These records encompassed physician encounters for pediatric patients presenting to this institution from January 2016 to July 2017. The median age was 2.35 years (range: 0 to 18, 95% confidence interval: 0.2 to 9.7 years old), and 40.11% were female (Table 1). 11,926 patient visit records from an independent cohort of pediatric patients from Zhengcheng Women and Children's Hospital (Guangdong Province, China) were used for a comparison study between the present AI system and human physicians.

The study was approved by the Guangzhou Women and Children's Medical Center and Zhengcheng Women and Children's Hospital institutional review board and ethics committee and complied with the Declaration of Helsinki. Consents were obtained from all participants at the initial hospital visit. Patient sensitive information was removed during the initial extraction of EHR data and EHR were de-identified. A data use agreement was composed and upheld by all institutions involved in the data collection and analysis. Data were stored in a fully HIPAA-compliant manner.

Inpatient disease prevalence from Table 1 is derived from the official government statistics report from the Guangdong province. Nursing flowsheets, such as the medication administration record, were not included. All encounters were labeled with a primary diagnosis in the International Classification of Disease ICD-10 coding that was determined by the examining physician.

TABLE 1
General characteristics of the study cohort. Characteristics for the patients
whose encounters were documented in the electronic health record (EHR)
and included in the training and testing cohorts for the analysis.

| | Cantonese | | | | |
|---|---|---|---|---|---|
| Disease | population | | Training | Testing | |
| group | incidence rate | Gender and age | Cohort | Cohort | Total |
| | | Males | 571,080 | 244,839 | 815,919 |
| | | | (59.87%) | (59.90%) | (59.88%) |
| | | Females | 382,709 | 163,931 | 546,640 |
| | | | (40.13%) | (40.10%) | (40.11%) |
| | | Median Age at Visit | 2.35 | 2.36 | 2.35 |
| | | (years) | | | |
| | | Most Common | | | |
| | | Primary Diagnoses | | | |
| Respiratory | 38.00% | Acute upper | 144,754 | 338,300 | 483,054 |
| disease | | respiratory infection | | | |
| | | Bronchitis (acute or | 123,447 | 286,393 | 409,840 |
| | | chronic) | | | |
| | | Acute bronchiolitis | 14,092 | 33,002 | 47,094 |
| | | Bronchopneumonia | 13,359 | 30,870 | 44,229 |
| | | Acute sinusitis | 8,606 | 20,131 | 28,737 |
| | | Acute tonsillitis | 10,487 | 24,077 | 34,564 |
| Digestive | 11.10% | Infectious gastroenteritis | 7,756 | 17,690 | 25,446 |
| disease | | and colitis | | | |
| | | Diarrhea | 16,337 | 38,267 | 54,604 |
| | | Enteroviral vesicular | 8,864 | 20,771 | 29,635 |
| | | stomatitis with exanthem | | | |
| | | Most Common | | | |
| | | Departments | | | |
| | | Represented | | | |
| | | General Pediatrics | 693,596 | 297,491 | 991,087 |
| | | Special Clinic for | 74,954 | 32,147 | 107,101 |
| | | Children | | | |
| | | Pediatric Pulmonology | 53,885 | 23,204 | 77,089 |
| | | Pediatric Emergency | 48,820 | 20,900 | 69,720 |
| | | Medicine | | | |
| | | Otolaryngology | 19,943 | 8,529 | 28,472 |
| | | Infectious Disease Clinic | 17,096 | 7,260 | 24,356 |
| | | Pediatric Rehabilitation | 12,977 | 5,348 | 18,325 |
| | | Neonatology | 8,234 | 3,505 | 11,739 |
| | | Pediatric | 8,067 | 3,599 | 11,666 |
| | | Gastroenterology | | | |

The primary diagnoses included 55 diagnosis codes encompassing common diseases in pediatrics and representing a wide range of pathology. Some of the most frequently encountered diagnoses included acute upper respiratory infection, bronchitis, diarrhea, bronchopneumonia, acute tonsillitis, stomatitis, and acute sinusitis (Table 1). The records originated from a wide range of specialties, with the top three most represented departments being general pediatrics, the Special Clinic for Children, and pediatric pulmonology (Table 1). The Special Clinic for Children consisted of a specific clinic for private or VIP patients at this institution and encompassed care for a range of conditions.

(A) NLP Model Construction

An information extraction model was established, which extracted the key concepts and associated categories in EHR raw data and transformed them into reformatted clinical data in query-answer pairs (FIG. 4). The reformatted chart grouped the relevant symptoms into categories, which increased transparency by showing the exact features that the model relies on to make a diagnosis. The schemas had been curated and validated by three physicians, which encompassed chief complaint, history of present illness, physical examination, and lab reports. There were multiple components to the NLP framework: 1) lexicon construction, 2) tokenization, 3) word embedding, 4) schema construction, and 5) sentence classification using Long Short Term Memory (LSTM) architecture.

Lexicon Construction

The lexicon was generated by manually reading sentences in the training data (approximately 1% of each class, consisting of over 11,967 sentences) and selecting clinically relevant words for the purpose of query-answer model construction. The keywords were curated by physicians and were generated by using a Chinese medical dictionary, which is analogous to the Unified Medical Language System (UMLS) in the United States. Next, any errors in the lexicon were revised according to physicians' clinical knowledge and experience, as well as expert consensus guidelines, based on conversations between board-certified internal medicine physicians, informaticians, and one health information management professional. This procedure was iteratively conducted until no new concepts of history of present illness (HPI) and physical exam (PE) were found.

Schema Design

A schema is a type of abstract synthesis of medical knowledge and physician experience, which is fixed in the form of certain rules. Once the schema is fixed, the information that natural language processing can obtain from the medical records is also fixed.

A schema is a group of three items <item_name, key_location, value>. The item_name is the feature name. The key_location encodes anatomical locations. The value consists of either free text or a binary number depending on the query type. When doing pattern matching, the NLP results were assessed to check if they could match to certain schema, and the results were filled out to the fourth column of the form while the first three columns remained unchanged.

Four information schemas were constructed with the curation of three physicians: history of present illness, physical examination, laboratory tests, and radiology reports (Supplementary Table 1). The chief complaint and history of present illness shared the same schema. The information contained in the schemas is shown in Supplementary Table 1.

Tokenization and Word Embedding

Due to the lack of publicly available community annotated resources for the clinical domain in Chinese, standard datasets for word segmentation were generated. The tool used for tokenization was mecab (url: https://github.com/taku910/mecab), with the curated lexicons described herein as the optional parameter. There were a total of 4363 tokens. Word2vec from python Tensorflow package was used to embed the 4363 tokens with 100 high dimensional features.

LSTM Model Training Data Set and Testing Data Set Construction

A small set of data was curated for training the text classification model. The query-answer pair in the training (n=3564) and validation (n=2619) cohort were manually annotated. For questions with binary answers, 0/1 were used to indicate if the text gave a no/yes. For example, given the text snippet “patient has fever”, query “is patient having fever?” will be assigned a value of 1. For queries with categorical/numerical values, the pre-defined categorical free text answer was extracted as shown in the schema (Supplementary Table 1).

The free-text harmonization process was modeled by the attention-based LSTM described in Luong et al. 20151. The model was implemented using tensorflow and trained with 200,000 steps. The NLP model was applied to all the physician notes, which were converted into the structured format (e.g., machine readable format), where each structured record contained data in query-answer pairs.

The hyperparameters were not tuned, and instead either default or commonly used settings of hyperparameters were used for the LSTM model. A total of 128 hidden units per layer and 2 layers of LSTM cells were used along with a default learning rate of 0.001 from Tensorflow.

(B) Hierarchical Multi-Label Diagnosis Model Construction

Diagnosis Hierarchy Curation

The relationship between the labels was curated by one US board-certified physician and two Chinese board-certified physicians. An anatomically based classification was used for the diagnostic hierarchy, as this was a common method of formulating a differential diagnosis when a human physician evaluates a patient. First, the diagnoses were separated into general organ systems (e.g. respiratory, neurologic, gastrointestinal, etc.). Within each organ system, there was a subdivision into subsystems (e.g. upper respiratory and lower respiratory). A separate category was labeled “generalized systemic” in order to include conditions that affected more than one organ system and/or were more generalized in nature (e.g. mononucleosis, influenza).

Model Training and Validation Process

The data was split into a training cohort, consisting of 70% of the total visit records, and a testing cohort, comprised of the remaining 30%. The feature space was then encoded as a visit by constructing a query-answer membership matrix for both the testing and training cohorts.

For each intermediate node, a multiclass linear logistic regression classifier was trained based on the immediate children terms. All the subclasses of the children terms were collapsed to the level of the children level. The one versus rest multiclass classifier was trained using Sklearn class LogisticRegression. A regularization penalty of 11 (Lasso) was also applied, simulating the case where physicians often rely on a limited number of symptoms to diagnose. The inputs were in query-answer pairs as described above. To further evaluate the model, the Receiver Operating Characteristic-Area Under Curves (ROC-AUC) (Supplementary Table 5) were also generated to evaluate the sensitivity and specificity of our multiclass linear logistic regression classifiers. The robustness of the classification models were also evaluated using a 5-fold cross-validation (Supplementary Table 6). The inputs were in query-answer pairs as described above.

SUPPLEMENTARY TABLE 5
ROC-AUC for each classification class in each classification group.
The multi-classification diagnosis models are composed of binary
classifiers and thus can also be evaluated in terms of ROC-AUC.

| Classification group | Classes | ROC-AUC |
|---|---|---|
| Asthma | Cough variant asthma | 0.964 |
| | Unspecified asthma with | 0.996 |
| | (acute) exacerbation | |
| | Unspecified asthma, | 0.975 |
| | uncomplicated | |
| Bacterial pneumonia | Bacterial pneumonia, not | 0.848 |
| | elsewhere classified | |
| | Bronchopneumonia | 0.848 |
| Encephalitis | Unspecified viral encephalitis | 0.869 |
| | Sequelae of viral encephalitis | 0.869 |
| GI | Acute pharyngitis | 0.939 |
| | Mouth-related Diseases | 0.942 |
| | Diarrhea, unspecified | 0.996 |
| Mouth-related Diseases | Stomatitis and related lesions | 0.973 |
| | Acute sialoadenitis | 0.979 |
| | Diseases of lips | 0.993 |
| | Acute tonsillitis, unspecified | 0.974 |
| Group: “Acute laryngitis” | Acute laryngotracheitis | 0.907 |
| | Acute laryngitis | 0.876 |
| | Acute laryngopharyngitis | 0.972 |
| Group: “Bronchitis” | Bronchiolitis | 0.965 |
| | Acute bronchitis, unspecified | 0.965 |
| Group: “Pneumonia” | Mycoplasma infection, | 0.952 |
| | unspecified site | |
| | Bacterial pneumonia | 0.952 |
| Group: “Sinusitis” | Acute recurrent sinusitis, | 0.977 |
| | unspecified | |
| | Acute sinusitis, unspecified | 0.977 |
| Lower Respiratory | Asthma | 0.973 |
| | Group: “Bronchitis” | 0.887 |
| | Group: “Pneumonia” | 0.927 |
| | Acute tracheitis | 0.976 |
| Neuro-Psych | Convulsions | 0.989 |
| | Bacterial meningitis, | 0.984 |
| | unspecified | |
| | Encephalitis | 0.977 |
| | Tic disorder, unspecified | 0.989 |
| | Attention-deficit | 0.981 |
| | hyperactivity disorders | |
| Respiratory | Lower Respiratory | 0.973 |
| | Upper respiratory | 0.973 |
| Upper respiratory | Group: “Sinusitis” | 0.994 |
| | Acute upper respiratory | 0.978 |
| | infection, unspecified | |
| | Group: “Acute laryngitis” | 0.986 |
| root | Systemic-Generalized | 0.968 |
| | Neuro-Psych | 0.996 |
| | GI | 0.972 |
| | Respiratory | 0.977 |
| | Genitourinary | 0.983 |
| Systemic-Generalized | Influenza | 0.989 |
| | Sepsis, unspecified organism | 0.985 |
| | Exanthema subitum [sixth | 0.995 |
| | disease] | |
| | Hand, foot, and mouth | 0.989 |
| | disease | |
| | Infectious mononucleosis | 0.99 |
| | Varicella without | 0.993 |
| | complication | |

SUPPLEMENTARY TABLE 6
Illustration of the diagnostic performance of the logistic regression
classifier at multiple levels of the diagnostic hierarchy with
5-fold cross-validation. The classification performance of
each diagnosis level is listed on each row. The classification
performance of each fold is listed in each column.

| | | | | | | Median |
|---|---|---|---|---|---|---|
| | | | | | | accuracy |
| | Folds | | | | | across |
| | 0 | 1 | 2 | 3 | 4 | folds |
| Asthma | 0.886 | 0.934 | 0.927 | 0.913 | 0.899 | 0.913 |
| Encephalitis | 0.83 | 0.771 | 0.809 | 0.868 | 0.848 | 0.83 |
| GI | 0.824 | 0.749 | 0.807 | 0.839 | 0.846 | 0.824 |
| Mouth-related | 0.865 | 0.899 | 0.857 | 0.884 | 0.915 | 0.884 |
| Diseases | | | | | | |
| Group: | 0.881 | 0.905 | 0.885 | 0.916 | 0.887 | 0.887 |
| “Bronchitis” | | | | | | |
| Group: | 0.838 | 0.804 | 0.92 | 0.916 | 0.861 | 0.861 |
| “Pneumonia” | | | | | | |
| Group: “Sinusitis” | 0.874 | 0.952 | 0.925 | 0.925 | 0.882 | 0.925 |
| Neuro-Psych | 0.849 | 0.869 | 0.864 | 0.862 | 0.87 | 0.864 |
| Respiratory | 0.778 | 0.934 | 0.938 | 0.934 | 0.916 | 0.934 |
| Upper respiratory | 0.844 | 0.891 | 0.918 | 0.928 | 0.932 | 0.918 |
| root | 0.786 | 0.884 | 0.886 | 0.88 | 0.861 | 0.88 |
| Systemic- | 0.905 | 0.922 | 0.897 | 0.904 | 0.915 | 0.905 |
| Generalized | | | | | | |
| | | | | | Median | 0.885 |
| | | | | | accuracy | |
| | | | | | across | |
| | | | | | folds and | |
| | | | | | classes | |

Hierarchical Clustering of Disease

The mean profile of the feature membership matrix was correlated using Pearson correlation. Hierarchical clustering was done by clustermap function from python seaborn package with default parameters.

To evaluate the robustness of the clustering result (FIG. 1), the data was first split into training and test sets by half and regenerated the two cluster maps for the training and test data independently. The leaves in both the training and test cluster maps were assigned to ten classes by cutting the associated dendrogram at the corresponding height independently. The class assignment concordance between the training and test data was evaluated by the Adjusted Rand Index (ART). An ARI value closer to 1 indicates higher concordance between training class assignment and test class assignment, whereas an ARI closer to 0 indicates close to the null background. A high ARI of 0.8986 between the training and test class assignments was observed, suggesting that the cluster map is robust.

Comparative Performance Between Our AI System and Human Physicians

A comparison study between the present AI system versus human physicians was conducted using 11,926 records from an independent cohort of pediatric patients from Zhengcheng Women and Children's Hospital, Guangdong Province, China. 20 pediatricians in five group with increasing levels of proficiency and years of clinical practice experience (4 in each level) were chosen to manually grade 11,926 records. These five groups are: senior resident physicians with more than three-year practice experience, junior physicians with eight-year practice experience, mid-level physicians with 15-year practice experience, attending physicians with 20-year practice experience, senior attending physicians with more than 25-year practice experience. A physician in each group read a random subset of 2981 clinical notes from this independent validation dataset and assigned a diagnosis. Each patient record was randomly assigned and graded by four physicians (one in each physician group). The diagnostic performance of each physician group in each of top 15 diagnosis categories was evaluated using an F1-score (Table 4)

Results

Unsupervised Diagnosis Grouping

First, the diagnostic system analyzed the EHR in the absence of a defined classification system with human input. In the absence of pre-defined labeling, the computer was still able to detect trends in clinical features to generate a relatively sensible grouping structure (FIG. 1). In several instances, the computer clustered together diagnoses with related ICD-10 codes, illustrating that it was able to detect trends in clinical features that align with a human-defined classification system. However, in other instances, it clustered together related diagnoses but did not include other very similar diagnoses within this cluster. For example, it clustered “asthma” and “cough variant asthma” into the same cluster, but it did not include “acute asthma exacerbation,” which was instead grouped with “acute sinusitis”. Several similar pneumonia-related diagnosis codes were also spread across several different clusters instead of being grouped together. However, in many instances, it successfully established broad grouping of related diagnoses even without any directed labeling or classification system in place.

Medical Record Reformatting Using NLP

A total of 6,183 charts were manually annotated using the schema described in the Methods section by senior attending physicians with more than 15 years clinical practice experience. The 3,564 manually annotated charts were then used to train the NLP information extraction model, and the remaining 2,619 were used to validate the model. The information extraction model summarized the key conceptual categories representing clinical data (FIG. 2). This NLP model utilized deep learning techniques (see Methods) to automate the annotation of the free text EHR notes into the standardized lexicon and clinical features allowing the further processing for diagnostic classification.

The median number of records included in the training cohort for any given diagnosis was 1,677, but there was a wide range (4 to 321,948) depending on the specific diagnosis. Similarly, the median number of records in the test cohort for any given diagnosis was 822, but the number of records also varied (range: 3 to 161,136) depending on the diagnosis.

The NLP model achieved excellent results in the annotation of the EHR physician notes (Table 2). Across all categories of clinical data, e.g., chief complaint, history of present illness, physical examination, laboratory testing, and PACS (Picture Archiving and Communication System) reports, the F1 scores exceeded 90% except in one instance, which was for categorical variables detected in laboratory testing. The highest recall of the NLP model was achieved for physical examination (95.62% for categorical variables, 99.08% for free text), and the lowest for laboratory testing (72.26% for categorical variables, 88.26% for free text). The precision of the NLP model was highest for chief complaint (97.66% for categorical variables, 98.71% for free text), and lowest for laboratory testing (93.78% for categorical variables, and 96.67% for free text). In general, the precision (or positive predictive value) of the NLP labeling was slightly greater than the recall (the sensitivity), but the system demonstrated overall strong performance across all domains (Table 2).

TABLE 2
Performance of the natural language processing (NLP) model. The performance
of the deep learning-based NLP model in annotating the physician-patient
encounters based on recall, precision, F1 scores, and instances of exact
matches are detailed here for each category of clinical data.

| Category of | | | | | | Exact |
|---|---|---|---|---|---|---|
| Clinical Data | Type | Count | Recall | Precision | F1 Score | Match |
| Chief Complaint | Classification | 23,130 | — | — | — | 98.14% |
| | Categorical | 71 | 83.19% | 97.66% | 89.85% | 52.11% |
| | Variables | | | | | |
| | Free Text | 10,416 | 97.92% | 98.71% | 98.31% | 96.58% |
| History of Present | Classification | 147,725 | — | — | — | 98.92% |
| Illness | Categorical | 1,434 | 89.09% | 94.05% | 91.50% | 72.66% |
| | Variables | | | | | |
| | Free Text | 25,352 | 96.24% | 96.73% | 96.49% | 92.67% |
| Physical | Classification | 170,075 | — | — | — | 99.48% |
| Examination | Categorical | 1,900 | 95.62% | 96.22% | 95.92% | 90.26% |
| | Variables | | | | | |
| | Free Text | 85,315 | 99.08% | 99.30% | 99.19% | 98.41% |
| Laboratory | Classification | 19,365 | — | — | — | 96.32% |
| Testing | Categorical | 456 | 72.26% | 93.78% | 81.62% | 54.17% |
| | Variables | | | | | |
| | Free Text | 9,407 | 88.26% | 96.67% | 92.27% | 93.27% |
| PACS Reports | Classification | 69,346 | — | — | — | 96.02% |
| | Categorical | 1,521 | 91.02% | 95.08% | 93.00% | 73.24% |
| | Variables | | | | | |
| | Free Text | 16,751 | 95.64% | 95.13% | 95.38% | 89.56% |

Performance of the Hierarchical Diagnosis Model

After the EHR notes were annotated using the deep NLP information extraction model, logistic regression classifiers were used to establish a hierarchical diagnostic system. The diagnostic system was primarily based on anatomic divisions, e.g. organ systems. This was meant to mimic traditional frameworks used in physician reasoning in which an organ-based approach can be employed for the formulation of a differential diagnosis. Logistic regression classifiers were used to allow straightforward identification of relevant clinical features and ease of establishing transparency for the diagnostic classification.

The first level of the diagnostic system categorized the EHR notes into broad organ systems: respiratory, gastrointestinal, neuropsychiatric, genitourinary, and generalized systemic conditions (FIG. 3). This was the first level of separation in the diagnostic hierarchy. Then, within each organ system, further sub-classifications and hierarchical layers were made where applicable. The most number of diagnoses in this cohort fell into the respiratory system, which was further divided into upper respiratory conditions and lower respiratory conditions. These were further separated into more specific anatomic divisions (e.g. laryngitis, tracheitis, bronchitis, pneumonia) (see Methods). The performance of the classifier was evaluated at each level of the diagnostic hierarchy. In short, the system was designed to evaluate the extracted features of each patient record and categorize the set of features into finer levels of diagnostic specificity along the levels of this decision tree, similar to how a human physician might evaluate a patient's features to achieve a diagnosis based on the same clinical data incorporated into the information model. Encounters labeled by physicians as having a primary diagnosis of “fever” or “cough” were eliminated, as these represented symptoms rather than specific disease entities.

Across all levels of the diagnostic hierarchy, this diagnostic system achieved a high level of accuracy between the predicted primary diagnoses based on the extracted clinical features by the NLP information model and the initial diagnoses designated by the examining physician (Table 3). For the first level where the diagnostic system classified the patient's diagnosis into a broad organ system, the median accuracy was 0.90, ranging from 0.85 for gastrointestinal diseases to 0.98 for neuropsychiatric disorders (Table 3a). Even at deeper levels of diagnostic specification, the system retained a strong level of performance. To illustrate, within the respiratory system, the next division in the diagnostic hierarchy was between upper respiratory and lower respiratory conditions. The system achieved an accuracy of 0.89 of upper respiratory conditions and 0.87 of lower respiratory conditions between predicted diagnoses and initial diagnoses (Table 3b). When dividing the upper respiratory subsystem into more specific categories, the median accuracy was 0.92 (range: 0.86 for acute laryngitis to 0.96 for sinusitis, Table 3c). Acute upper respiratory infection was the single most common diagnosis among the cohort, and the model was able to accurately predict the diagnosis in 95% of the encounters (Table 3c). Within the respiratory system, asthma was categorized separately as its own subcategory, and the accuracy ranged from 0.83 for cough variant asthma to 0.97 for unspecified asthma with acute exacerbation (Table 3d).

Table 3. Illustration of diagnostic performance of the logistic regression classifier at multiple levels of the diagnostic hierarchy. A) At the first level of the diagnostic hierarchy, the framework accurately discerned broad anatomic classifications between organ systems in this large cohort of pediatric patients. For example, among 315,661 encounters with primary respiratory diagnoses as determined by human physicians, the computer was able to correctly predict the diagnoses in 295,403 (92%) of them. B) Within the respiratory system, at the next level of the diagnostic hierarchy, the framework could discern between upper respiratory conditions and lower respiratory conditions. C) Within the upper respiratory system, further distinctions could be made into acute upper respiratory infection, sinusitis, and laryngitis. Acute upper respiratory infection and sinusitis were among the most common conditions in the entire cohort, and diagnostic accuracy exceeded 95% in both entities. D) Asthma was categorized as a separate category within the respiratory system, and the diagnostic system accurately distinguished between uncomplicated asthma, cough variant asthma, and acute asthma exacerbation.

TABLE 3A

| | Physician-Assigned Diagnoses | | | | |
|---|---|---|---|---|---|
| Computer- | | Gastro- | Systemic | | |
| Predicted | Respiratory | intestinal | Generalized | Neuropsychiatric | Genitourinary |
| Diagnoses | (n = 315,661) | (n = 41,098) | (n = 11,698) | (n = 8,410) | (n = 1,326) |
| Respiratory | 0.92 | 0.1 | 0.048 | 0.0052 | 0.049 |
| (n = 295,403) | | | | | |
| Gastrointestinal | 0.063 | 0.85 | 0.066 | 0.0052 | 0.044 |
| (n = 55,704) | | | | | |
| Systemic/ | 0.009 | 0.028 | 0.87 | 0.0034 | 0.012 |
| Generalized | | | | | |
| (n = 14,267) | | | | | |
| Neuropsychiatric | 0.0018 | 0.0032 | 0.0034 | 0.98 | 0.0053 |
| (n = 9,007) | | | | | |
| Genitourinary | 0.0062 | 0.014 | 0.0077 | 0.0044 | 0.89 |
| (n = 3,812) | | | | | |

TABLE 3B

| | Physician-Assigned Diagnoses | |
|---|---|---|
| | Upper | Lower |
| Computer-Predicted | Respiratory | Respiratory |
| Diagnoses | (n = 156,176) | (n = 159,485) |
| Upper Respiratory (n = 158,890) | 0.89 | 0.11 |
| Lower Respiratory (n = 156,771) | 0.13 | 0.87 |

TABLE 3C

| | Physician-Assigned Diagnoses | | |
|---|---|---|---|
| | Acute upper | | |
| | respiratory | | Acute |
| Computer-Predicted | infection | Sinusitis | laryngitis |
| Diagnoses | (n = 144,503) | (n = 8,828) | (n = 2845) |
| Acute upper respiratory | 0.95 | 0.033 | 0.11 |
| infection (n = 137,995) | | | |
| Sinusitis (n = 10,859) | 0.016 | 0.96 | 0.028 |
| Acute laryngitis (n = 7,322) | 0.033 | 0.01 | 0.86 |

TABLE 3D

| | Physician-Assigned Diagnoses | | |
|---|---|---|---|
| | Uncomplicated | Cough variant | Acute asthma |
| Computer-Predicted | asthma | asthma | exacerbation |
| Diagnoses | (n = 776) | (n = 201) | (n = 121) |
| Uncomplicated | 0.91 | 0.16 | 0 |
| asthma (n = 740) | | | |
| Cough variant | 0.085 | 0.83 | 0.033 |
| asthma (n = 236) | | | |
| Acute asthma | 0.0039 | 0.01 | 0.97 |
| exacerbation (n = 122) | | | |

In addition to the strong performance in the respiratory system, the diagnostic model performed comparably in the other organ subsystems (see Supplementary Tables 1-4). Notably, the classifier achieved a very high level of association between predicted diagnoses and initial diagnoses for the generalized systemic conditions, with a accuracy of 0.90 for infectious mononucleosis, 0.93 for roseola (sixth disease), 0.94 for influenza, 0.93 for varicella, and 0.97 for hand-foot-mouth disease (Supplementary Table 4). The diagnostic framework also achieved high accuracy for conditions with potential for high morbidity, such as bacterial meningitis, for which the accuracy between computer-predicted diagnosis and physician-assigned diagnosis was 0.93 (Supplementary Table 3).

Supplementary Table 1. Diagnostic performance in the gastrointestinal system. A) The classifier performed with high accuracy across multiple entities grouped under the category of gastrointestinal diseases in this pediatric cohort. B) In the mouth-related disease category, the classifier exhibited a high level of correlation with physician-assigned diagnoses even for very specific entities.

| | SUPPLEMENTARY TABLE 1A | | |
|---|---|---|---|
| | Physician-Assigned Diagnoses | | |
| | Mouth-related | Acute | |
| Computer-Predicted | diseases | pharyngitis | Diarrhea |
| Diagnoses | (n = 13,315) | (n = 11,429) | (n = 16,354) |
| Mouth-related diseases | 0.86 | 0.11 | 0.016 |
| (n = 13,024) | | | |
| Acute pharyngitis | 0.12 | 0.87 | 0.0079 |
| (n = 11,774) | | | |
| Diarrhea (n = 16,300) | 0.012 | 0.016 | 0.98 |

| | SUPPLEMENTARY TABLE 1B | | | |
|---|---|---|---|---|
| | Physician-Assigned Diagnoses | | | |
| | | | Acute | Disease |
| | Acute | | siaload- | of the |
| Computer-Predicted | tonsillitis | Stomatitis | enitis | lips |
| Diagnoses | (n = 10,269) | (n = 2,596) | (n = 309) | (n = 141) |
| Acute tonsillitis | 0.94 | 0.065 | 0.084 | 0.0071 |
| (n = 9,874) | | | | |
| Stomatitis | 0.042 | 0.91 | 0.074 | 0.071 |
| (n = 2,836) | | | | |
| Acute sialoadenitis | 0.015 | 0.021 | 0.83 | 0.0071 |
| (n = 469) | | | | |
| Diseases of the lips | 0 | 0.0015 | 0.0097 | 0.91 |
| (n = 136) | | | | |

Supplementary Table 2. Diagnostic performance in respiratory system subgroups. a) The classifier could accurately distinguish between acute bronchitis and bronchiolitis, as well as b) between different types of pneumonia, demonstrating high performance even within very specific diagnoses.

SUPPLEMENTARY TABLE 2a

Physician-Assigned Diagnoses

| Computer-Predicted | Acute bronchitis | Bronchiolitis |
|---|---|---|
| Diagnoses | (n = 30,530) | (n = 14,152) |
| Acute bronchitis | 0.94 | 0.065 |
| (n = 29,727) | | |
| Bronchiolitis (n = 14,955) | 0.057 | 0.93 |

SUPPLEMENTARY TABLE 2b

Physician-Assigned Diagnoses

| | Bacterial | Mycoplasma |
|---|---|---|
| Computer-Predicted | Pneumonia | Pneumonia |
| Diagnoses | (n = 16,895) | (n = 1,926) |
| Bacterial Pneumonia | 0.89 | 0.13 |
| (n = 15,339) | | |
| Mycoplasma Pneumonia | 0.11 | 0.87 |
| (n = 3,482) | | |

SUPPLEMENTARY TABLE 3
Diagnostic performance in the neuropsychiatric system. The classifier performed
with generally high accuracy across disease entities in the neuropsychiatric
system. “Convulsions” included both epileptic conditions and febrile
convulsions, and performance may have been affected by the small sample size.

| | Physician-Assigned Diagnoses | | | | |
|---|---|---|---|---|---|
| | | Attention | | | |
| | | deficit | | | |
| | | hyperactivity | Bacterial | | |
| Computer-Predicted | Tic disorder | disorder | meningitis | Encephalitis | Convulsions |
| Diagnoses | (n = 4,625) | (n = 2,819) | (n = 516) | (n = 394) | (n = 56) |
| Tic disorder | 0.94 | 0.071 | 0.0097 | 0.028 | 0.054 |
| (n = 4,552) | | | | | |
| Attention deficit | 0.046 | 0.91 | 0.0078 | 0.015 | 0 |
| hyperactivity | | | | | |
| disorder (n = 2,792) | | | | | |
| Bacterial meningitis | 0.0067 | 0.0074 | 0.93 | 0.11 | 0.089 |
| (n = 580) | | | | | |
| Encephalitis (n = 384) | 0.0032 | 0.0053 | 0.039 | 0.84 | 0.071 |
| Convulsions (n = 102) | 0.0074 | 0.0046 | 0.014 | 0.01 | 0.79 |

SUPPLEMENTARY TABLE 4
Diagnostic performance among generalized systemic disorders. These diagnoses were included
for affecting multiple organ systems or for producing generalized symptoms.

| | Physician-Assigned Diagnoses | | | | | Exanthema |
|---|---|---|---|---|---|---|
| | Hand-foot- | | | | | subitum |
| Computer- | mouth | | | Infectious | | (sixth |
| Predicted | disease | Varicella | Influenza | mononucleosis | Sepsis | disease) |
| Diagnoses | (n = 8,987) | (n = 748) | (n = 691) | (n = 563) | (n = 406) | (n = 303) |
| Hand-foot-mouth | 0.97 | 0.024 | 0.0058 | 0.0071 | 0.037 | 0.0066 |
| disease (n = 8,789) | | | | | | |
| Varicella (n = 783) | 0.0075 | 0.93 | 0.0043 | 0.0071 | 0.0025 | 0.033 |
| Influenza (n = 815) | 0.01 | 0.012 | 0.94 | 0.059 | 0.054 | 0.02 |
| Infectious | 0.0032 | 0.0053 | 0.025 | 0.9 | 0.037 | 0.0099 |
| mononucleosis | | | | | | |
| (n = 577) | | | | | | |
| Sepsis (n = 390) | 0.0012 | 0.004 | 0.022 | 0.02 | 0.86 | 0 |
| Exanthema | 0.0045 | 0.021 | 0.0014 | 0.0036 | 0.0074 | 0.93 |
| subitum (sixth | | | | | | |
| disease) (n = 344) | | | | | | |

Identification of Common Features Driving Diagnostic Prediction

To gain insight into how the diagnostic system generated a predicted diagnosis, we identified key clinical features driving the diagnosis prediction. For each feature, we identified what category of EHR clinical data it was derived from (e.g. history of present illness, physical exam, etc.) and whether it was coded as a binary classification or categorical. The interpretability of the predictive impact of used in the diagnostic system allowed the evaluation of whether the prediction was based on clinically relevant features.

For instance, using gastroenteritis as an example, the diagnostic system identified words such as “abdominal pain” and “vomiting” as key associated clinical features. The binary classifiers were coded such that the presence of a feature was denoted as “1” and absence was denoted as “0”. In this case, “vomiting=1” and “abdominal pain=1” were identified as key features for both chief complaint and history of present illness. Under physical exam, “abdominal tenderness=1” and “rash=1” were noted to be associated with this diagnosis. Interestingly, “palpable mass=0” was also associated, meaning that the patients predicted to have gastroenteritis usually did not have a palpable mass, which is consistent with human clinical experience. In addition to binary classifiers, there were also nominal categories in the schema. The feature of “fever” with a text entry of greater than 39 degrees Celsius also emerged as an associated clinical feature driving the diagnosis of gastroenteritis. Laboratory and imaging features were not identified as strongly driving the prediction of this diagnosis, perhaps reflecting the fact that most cases of gastroenteritis are diagnosed without extensive ancillary tests.

AI Comparison to Human Physicians

The performance of the diagnosis between the AI model and human physicians was compared using 11,926 records from an independent cohort of pediatric patients. Twenty pediatricians in five groups with increasing levels of proficiency and years of clinical practice experience (see method section for description) manually graded 11,926 records. A physician in each group read a random subset of the raw clinical notes from this independent validation data and assigned a diagnosis. Next, the diagnostic performance of each physician group in each of top 15 diagnosis categories was evaluated using an F1-score (Table 4). Our model achieved an average F1-score higher than the two junior physician groups but lower than the three senior physician groups. This result suggests that this AI model may potentially assist junior physicians in diagnosis.

TABLE 4
Illustration of diagnostic performance between our AI model and
physicians. F1-score was used to evaluate the diagnosis performance
across different diagnosis groups (rows) between the model, and
two junior physician groups and three senior physician groups
(columns, see method section for description). It was observed
that the model performed better than junior physician groups but
slightly worse than three experienced physician groups.

| | | Physicians | | | | |
|---|---|---|---|---|---|---|
| | | Physi- | Physi- | Physi- | Physi- | Physi- |
| | | cian | cian | cian | cian | cian |
| | Our | group | group | group | group | group |
| | model | #1 | #2 | #3 | #4 | #5 |
| Asthma | 0.92 | 0.801 | 0.837 | 0.904 | 0.89 | 0.935 |
| Encephalitis | 0.837 | 0.947 | 0.961 | 0.95 | 0.959 | 0.965 |
| GI | 0.865 | 0.818 | 0.872 | 0.854 | 0.896 | 0.893 |
| Group: | 0.786 | 0.808 | 0.73 | 0.879 | 0.94 | 0.943 |
| “Acute | | | | | | |
| laryngitis” | | | | | | |
| Group: | 0.888 | 0.829 | 0.767 | 0.946 | 0.952 | 0.972 |
| “Pneumonia” | | | | | | |
| Group: | 0.932 | 0.839 | 0.797 | 0.896 | 0.873 | 0.87 |
| “Sinusitis” | | | | | | |
| Lower | 0.803 | 0.803 | 0.815 | 0.91 | 0.903 | 0.935 |
| Respiratory | | | | | | |
| Mouth- | 0.897 | 0.818 | 0.872 | 0.854 | 0.896 | 0.893 |
| related | | | | | | |
| Diseases | | | | | | |
| Neuro-Psych | 0.895 | 0.925 | 0.963 | 0.96 | 0.962 | 0.906 |
| Respiratory | 0.935 | 0.808 | 0.769 | 0.89 | 0.907 | 0.917 |
| Systemic- | 0.925 | 0.879 | 0.907 | 0.952 | 0.907 | 0.944 |
| Generalized | | | | | | |
| Upper | 0.929 | 0.817 | 0.754 | 0.884 | 0.916 | 0.916 |
| respiratory | | | | | | |
| root | 0.889 | 0.843 | 0.863 | 0.908 | 0.903 | 0.912 |
| Average F- | 0.885 | 0.841 | 0.839 | 0.907 | 0.915 | 0.923 |
| score | | | | | | |

Discussion

In this study, an artificial intelligence (AI)-based natural language processing (NLP) model was generated which could process free text from physician notes in the electronic health record (EHR) to accurately predict the primary diagnosis in a large pediatric population. The model was initially trained by a set of notes that were manually annotated by an expert team of physicians and informatics researchers. Once trained, the NLP information extraction model used deep learning techniques to automate the annotation process for notes from over 1.4 million encounters (pediatric patient visits) from a single institution in China. With the clinical features extracted and annotated by the deep NLP model, logistic regression classifiers were used to predict the primary diagnosis for each encounter. This system achieved excellent performance across all organ systems and subsystems, demonstrating a high level of accuracy for its predicted diagnoses when compared to the initial diagnoses determined by an examining physician.

This diagnostic system demonstrated particularly strong performance for two important categories of disease: common conditions that are frequently encountered in the population of interest, and dangerous or even potentially life-threatening conditions, such as acute asthma exacerbation and meningitis. Being able to predict common diagnoses as well as dangerous diagnoses is crucial for any diagnostic system to be clinically useful. For common conditions, there is a large pool of data to train the model, so this diagnostic system is expected to exhibit better performance with more training data. Accordingly, the performance of the diagnostic system described herein was especially strong for the common conditions of acute upper respiratory infection and sinusitis, both which had an accuracy of 0.95 between the machine-predicted diagnosis and the human-generated diagnosis. In contrast, dangerous conditions tend to be less common and would have less training data. Despite this, a key goal for any diagnostic system is to achieve high accuracy for these dangerous conditions in order to promote patient safety. The present diagnostic system was able to achieve this in several disease categories, as illustrated by its performance for acute asthma exacerbations (0.97), bacterial meningitis (0.93) and across multiple diagnoses related to systemic generalized conditions, such as varicella (0.93), influenza (0.94), mononucleosis (0.90), and roseola (0.93). These are all conditions that can have potentially serious and sometimes life-threatening sequelae, so accurate diagnosis is of utmost importance.

In addition to its diagnostic accuracy, this system featured several other key strengths. One was that it allowed visualization of clinical features used for establishing the diagnosis. A key concern of AI-based methods in medicine is the “black box” nature of the analysis, but here the present approach provided the identification of the key clinical features for each diagnosis. This transparency allowed confirmation that the features being used by the deep-learning based model were clinically relevant and aligned with what human physicians have identified as important distinguishing or even pathognomonic features for diagnosis. Another strength of this study was the massive volume of data that was used, with over 1.4 million records included in the analysis. The large volume of encounters contributed to the robustness of the diagnostic system. Furthermore, another strength was that the data inputs in this model were harmonized. This represents an unconventional improvement upon other techniques, such as mapping the attributes to a fixed format (FHIR). Harmonized inputs describe the data in a consistent fashion and improve the quality of the data using machine learning capabilities. These strengths of transparency, high volume of data, and harmonization of data inputs are key advantages of this model compared with other NLP frameworks that have been previously reported.

Our overall framework of automating the extraction of clinical data concepts and features to facilitate diagnostic prediction can be applied across a wide array of clinical applications. The present study used primarily an anatomical or organ systems-based approach to the diagnostic classification. This broad generalized approach is often used in the formulation of differential diagnoses by physicians. However, the present disclosure can be modified to carry out a pathophysiologic or etiologic approach (e.g. “infectious” vs. “inflammatory” vs. “traumatic” vs. “neoplastic” and so forth). The design of the diagnostic hierarchy decision tree can be adjusted to what is most appropriate for the clinical situation.

In conclusion, this study describes an AI framework to extract clinically relevant information from free text EHR notes to accurately predict a patient's diagnosis. The NLP information model is able to perform the information extraction with high recall and precision across multiple categories of clinical data, and when processed with a logistic regression classifier, is able to achieve high association between predicted diagnoses and initial diagnoses determined by a human physician. This type of framework is useful for streamlining patient care, such as in triaging patients and differentiating between those patients who are likely to have a common cold from those who need urgent intervention for a more serious condition. Furthermore, this AI framework can be used as a diagnostic aid for physicians and assist in cases of diagnostic uncertainty or complexity, thus not only mimicking physician reasoning but actually augmenting it as well. Although this impact may be most obvious in areas where healthcare providers are in relative shortage compared to the overall population, such as China, healthcare resources are in high demand worldwide, and the benefits of such a system are likely to be universal.

### Example 2

The study of Example 1 is carried out on a patient population including non-Chinese and non-pediatric patients. Because the study of Example 1 focused on pediatric patients, most of whom presented for acute care visits, longitudinal analysis over time was less relevant. However, because the present study includes non-pediatric patients, a single patient's various encounters into a single timeline are collated to generate additional insights, particularly for adult patients or patients with chronic diseases that need long term management over time. Thus, the present study includes non-Chinese patients for purposes of diversifying the sources of data used to train the model.

An AI framework is generated to extract clinically relevant information from free text EHR notes to accurately predict a patient's diagnosis. The NLP information model is able to perform the information extraction with high recall and precision across multiple categories of clinical data, and when processed with a logistic regression classifier, is able to achieve high association between predicted diagnoses and initial diagnoses determined by a human physician.

### Example 3

Various biases can create problems with developing a reliable and trustworthy diagnostic model. Different measures can be taken to handle be potential biases in the model such as the model of example 1. For example, different hospitals from different regions of China might use different dialect, or use different EHR systems to structure the data, which might confuse the NLP model when the model is trained only in a hospital from Guangdong. Other models for word embeddings can be used to reduce bias. For example, word2vec is known to suffer outlier effect in word counts during word embeddings construction which may be avoided by adopting sense2vec. The performance of using LSTM-RNN versus adopting conditional random fields neural network (CRF-RNN) in the diagnostic model is also evaluated.

### Example 4

The AI-assisted diagnostic system incorporating the machine learning models or algorithms described in examples 1-2 can be implemented to improve clinical practice in several ways. First, it could assist with triage procedures. For example, when patients come to the emergency department or to an urgent care setting, their vital signs, basic history, and physical exam obtained by a nurse or midlevel provider could be entered into the framework, allowing the algorithm to generate a predicted diagnosis. These predicted diagnoses could help to prioritize which patients should get seen first by a physician. Some patients with relatively benign or non-urgent conditions may even be able to bypass the physician evaluation altogether and be referred for routine outpatient follow-up in lieu of urgent evaluation. This diagnostic prediction would help ensure that physicians' time is dedicated to the patients with the highest and/or most urgent needs. By triaging patients more effectively, wait times for emergent or urgent care may decrease, allowing improved access to care within a healthcare system of limited resources.

Another potential application of this framework is to assist physicians with the diagnosis of patients with complex or rare conditions. While formulating a differential diagnosis, physicians often draw upon their own experiences, and therefore the differential may be biased toward conditions that they have seen recently or that they have commonly encountered in the past. However, for patients presenting with complex or rare conditions, a physician may not have extensive experience with that particular condition. Misdiagnosis may be a distinct possibility in these cases. Utilizing this AI-based diagnostic framework harnesses the power generated by data from millions of patients and would be less prone to the biases of individual physicians. In this way, a physician could use the AI-generated diagnosis to help broaden his/her differential and think of diagnostic possibilities that may not have been immediately obvious.

In practical terms, implementation of the models described herein in various clinical settings would require validation in the population of interest. Ongoing data would need to be collected and used for continuous training of the algorithm to ensure that it is best serving the needs of the local patient population. Essentially, a local benchmark can be established to establish a reference standard, similar to how clinical laboratories establish local reference standards for blood-based biomarkers.

### Example 5

Abstract

Artificial intelligence (AI) has emerged as a powerful tool to transform medical care and patient management. Here we created an end-to-end AI platform using natural language processing (NLP) and deep learning techniques to extract relevant clinical information from adult and pediatric electronic health records (EHRs). This platform was applied to 2.6 million medical records from 1,805,795 adult and pediatric patients to train and validate the framework, which captures common pediatric and adult disease classifications. We validated our results in independent external cohorts. In an independent evaluation comparing AI and human physician diagnosis, AI achieves high diagnostic accuracy comparable to human physician and can improve healthcare delivery by preventing unnecessary hospital stays and reducing costs and readmission rates. Therefore, this study provides a proof of concept for the feasibility of an AI system in accurate diagnosis and triage of common human diseases with increased hospital efficiency, resulting in improved clinical outcomes.

Introduction

Within the past few decades, advances in computer science have met a long-standing need for structured and organized clinical data by introducing electronic health records (EHRs). EHRs represent a massive repository of electronic data points containing a diverse array of clinical information. Current advantages include standardization of clinical documentation, improvement of communication between healthcare providers, ease of access to clinical records, and an overall reduction in systematic errors. Given their safety, efficacy, and ability to provide a higher standard of care, medical communities have been transitioning to EHRs within the past decade, but the reservoir of information they contain has remained unexploited. With the advent of data mining, EHRs have emerged as a valuable resource for machine learning algorithms given their ability to find associations between many clinical variables and outcomes. EHRs not only contain a preliminary diagnosis and treatment plans but other information modalities, such as patient demographics, health risk factors, and family history that have the potential to guide disease management and improve outcomes both at the individual and population levels.

Current medical practice often uses hypothetical-deductive reasoning to determine disease diagnosis. In a typical clinical encounter, the patient provides the physician with a chief complaint, usually consisting of a few symptoms with a history of onset. This information ‘input’ then prompts the physician to ask a subset of appropriately targeted questions, which further explores the chief complaint and help to narrow down the differential diagnoses. Each subset of questions will be dependent upon the information provided from the patient's previous answer. Additional inputs such as past medical history, family history, physical examination findings, laboratory tests and/or imaging studies act as independent variables, which the physician assesses to rule in or out certain diagnoses. Whereas a physician can weigh up a handful of variables, AI algorithms have the potential to rapidly and accurately assess the probabilistic effects of hundreds of variables to reach likely diagnoses. This would provide physicians with a valuable aid in the field of healthcare. Already, machine learning methods have demonstrated efficacy in image-based diagnoses, notably in radiology, dermatology, and ophthalmology. We devised a machine learning artificial intelligence (AI)-based platform to extract pertinent features from EHR clinical entries by natural language processing and reach probable diagnoses in both adult and pediatric patient populations in an ‘end-to-end’ manner. This platform achieved high diagnostic efficiency across a diverse disease spectrum while demonstrating comparable performance to experienced physicians.

Results

Patient Characteristics

A total of 2,612,114 EHR records (380,665 adult; 2,231,449 pediatric) from 1,085,795 patients (223,907 adult, 861,888 pediatric) were collected for analysis. The First Affiliated Hospital of Guangzhou Medical University (GMU 1), provided 333,672 EHRs from 186,745 adult patients for machine learning and internal validation purposes. Guangzhou Women and Children's Medical Center (GWCMC1), provided 1,516,458 EHRs from 552,789 outpatient and inpatient pediatric visits for machine learning and internal validation purposes. The resulting AI-platform was externally validated on 46,993 EHRs involving 37,162 adult patients from The Second Affiliated Hospital of Guangzhou Medical University (GMU 2). External validation in the pediatric populations was performed on 714,991 EHRs from 339,099 pediatric patients from Guangzhou Women and Children's Medical Center (GWCMC2) from a second site in a different city (ZhuHai city). The weighted mean age across adult cohorts was 54.99 years (SD: +/−17.28; range: 18-104; 50.30% female) (Table 7A). The weighted mean age across pediatric cohorts was 3.28 years (SD: 2.75; range: 0 to 18; 41.10% female, Table 7B). Table 8A-B shows the breakdown percentages of respective adult and pediatric disease classifications in the study cohorts. For all encounters, physicians classified primary diagnosis through the use of the International Classification of Disease ICD-10 codes (World Health Organisation), which were then grouped according to organ-based systems (See Methods). Twelve adult and six pediatric organ-based diagnostic classifications encompassed a wide range of pathology across adult and pediatric cohorts. Cancer, respiratory, and cardiovascular diseases were the most frequently encountered diagnoses in adults (Table 8A), while ear-nose-throat, respiratory, and gastrointestinal diseases most frequently occurred in pediatric populations (Table 8B).

TABLE 7A
General characteristics of the adult cohorts. Characteristics
for the patients across all cohorts used in both training
internal/external validations. Encounters were documented
in the electronic health record (EHR).

| | Training and | | |
|---|---|---|---|
| | Internal | External | |
| | Validations | Validations | Overall |
| Cohort | GMU 1 | GMU 2 | Combined |
| Mean Age +/− SD | 54.23 +/− | 58.81 +/− | 54.99 +/− |
| | 17.27 | 17.33 | 17.28 |
| Age Range | 18-104 | 18-104 | 18-104 |
| Males | 93,623 | 17,659 | 111,282 |
| | | | (49.70%) |
| Females | 93,122 | 19,503 | 112,625 |
| | | | (50.30%) |
| Number of Patients | 186,745 | 37,162 | 223,907 |
| Number of EHRs | 333,672 | 46,993 | 380,665 |

TABLE 7B
General characteristics of the pediatric cohorts. Characteristics
for the patients across all cohorts used in both training
internal/external validations. Encounters were documented
in the electronic health record (EHR).

| | Training and | | |
|---|---|---|---|
| | Internal | External | |
| | Validations | Validations | Overall |
| Cohort | GWCMC1 | GWCMC2 | combined |
| Mean Age +/− SD | 3.36 +/− 2.77 | 3.15 +/− 2.72 | 3.28 +/− 2.75 |
| Age Range | 0-18 | 0-18 | 0-18 |
| Males | 308,458 | 199,184 | 507,642 |
| | | | (58.90%) |
| Females | 214,331 | 139,915 | 354,246 |
| | | | (41.10%) |
| Number of Patients | 522,789 | 339,099 | 861,888 |
| Number of EHRs | 1,516,458 | 714,991 | 2,231,449 |

TABLE 8A
Overview of Primary Diagnoses Across Adult Cohorts. Breakdown of primary
organ-based diagnostic classifications by percentage across adult cohorts.
Free segmented text implemented for training and validation purposes
from electronic health records (EHRs) obtained from The First Affiliated
Hospital of Guangzhou Medical University (GMU 1) and The Second Affiliated
Hospital of Guangzhou Medical University (GMU 2).

| | GMU 1 | | GMU2 | Total | |
|---|---|---|---|---|---|
| Training and Testing | Train | Test | Test | Total | Percent |
| on Adult Cohorts | set | set | set | Charts | Ratio |
| Tumor or cancer disease | 120386 | 834 | 9544 | 130,764 | 34.35% |
| Respiratory disease | 82065 | 834 | 3944 | 86,843 | 22.81% |
| Cardiovascular disease | 24574 | 834 | 5177 | 30,585 | 8.03% |
| Gynecological and | 22561 | 834 | 2102 | 25,497 | 6.70% |
| Obstetric disease | | | | | |
| Neuropsychiatric disease | 15611 | 834 | 4585 | 21,030 | 5.52% |
| Gastrointestinal disease | 19190 | 834 | 11270 | 31,294 | 8.22% |
| Urological disease | 12926 | 834 | 2002 | 15,762 | 4.14% |
| Orthopedic disease | 9261 | 834 | 3679 | 13,774 | 3.62% |
| Endocrinologic and | 7269 | 834 | 1856 | 9,959 | 2.62% |
| metabolic disease | | | | | |
| Ear-Nose-Throat disease | 3828 | 834 | 870 | 5,532 | 1.45% |
| Nephrological disease | 3378 | 834 | 1389 | 5,601 | 1.47% |
| Ophthalmological disease | 2615 | 834 | 575 | 4,024 | 1.06% |
| Total | 323,664 | 10,008 | 46,993 | 380,665 | 100.00% |

TABLE 8B
Overview of Primary Diagnoses Across Pediatric Cohorts. Breakdown of primary
organ-based diagnostic classifications by percentage across pediatric cohorts.
Free segmented text implemented for training and validation purposes from
electronic health records (EHRs) obtained from separate Guangzhou Women
and Children's Medical Center cohorts (GWCMC1 and GWCMC2).

| | GWCMC1 | | GWCMC2 | Total | |
|---|---|---|---|---|---|
| Training and Testing | Train | Test | Test | Total | Percent |
| on Pediatric Cohorts | set | set | set | Charts | Ratio |
| Ear-Nose-Throat disease | 1084146 | 1000 | 520,295 | 1605441 | 71.95% |
| Respiratory disease | 234925 | 1000 | 112,475 | 348400 | 15.61% |
| Gastrointestinal disease | 95114 | 1000 | 34,998 | 131112 | 5.88% |
| General Systemic disease | 55620 | 1000 | 23,049 | 79669 | 3.57% |
| Neuropsychiatric disease | 31075 | 1000 | 20,813 | 52888 | 2.37% |
| Urological disease | 9578 | 1000 | 3,361 | 13939 | 0.62% |
| Total | 1,510,458 | 6,000 | 714,991 | 2,231,449 | 100.00% |

An End-to-End Approach for Building an AI Diagnostic Model

A diagnostic classifier (FIG. 5) was built using end-to-end deep learning. The model reviewed the following three parameters per patient visit; chief complaint, history of present illness, and picture archiving and communication system (PACS) reports. Given that all EHRs were obtained from Chinese cohorts, text segmentation was essential in Chinese NLP due to the lack of spacing that separates meaningful units of text. As such, a comprehensive Chinese medical dictionary and Jieba, an open-source general-purpose Chinese word/phrase segmentation software, were applied to each record in order to extract relevant medical text (FIG. 9). Segmented words were then fed into a word embedding layer, followed by a bi-directional long-short term memory (LSTM) neural network layer. A diagnosis was selected by combining the forward and backward directional outputs of the LSTM layers (FIG. 5). The model was trained end-to-end to obtain optimal model parameters for all layers without any feature engineering other than the initial word segmentation. No labor-intensive labeling of clinical text features was necessary to train the model. Details of the model design and justification are given in Methods.

Performance of Diagnosing Common Adult and Pediatric Conditions

Internal validations achieved high accuracies across all general disease categories. Average diagnostic efficiency for adults was 96.35% and ranged from 93.17% (Neuropsychiatric diseases) to 97.84% (Urological diseases) in the GMU1 internal validation test (FIG. 6A and Table 9A). The AUC of the micro-average ROC for adult classifications was 0.996 (FIG. 6B). Average diagnostic efficiency for pediatrics was 91.85% and ranged from 83.50% (Ear-Nose-Throat diseases) to 97.80% (Neuropsychiatric diseases) in GWCMC1 internal validation tests (FIG. 6C and Table 9B). The AUC of the micro-average ROC for pediatric classifications was 0.983 (FIG. 6D). Percent correct classification and model loss over time can be seen in FIG. 10A-FIG. 10D. To further explore the precision of the model, a binary comparison between upper and lower respiratory diseases was performed in both adult and pediatric cohorts. The model achieved an average accuracy of 91.30% for adults (Table 10A) and 86.71% for pediatric patients (Table 10B). Next we evaluated if our AI model could distinguish the phenotypes between four common upper respiratory diseases and four common lower respiratory diseases. Multiclass comparisons showed high accuracies, where the average diagnostic efficiency for common upper and lower respiratory diseases were 92.25% and 84.85% respectively (Table 11A-11B) The highest upper and lower respiratory disease diagnoses were sinusitis and asthma with accuracies of 96.30% and 90.90% respectively. Other respiratory diseases showed high diagnostic efficiency and can be seen in Table 11A-11B. We also saw a high average accuracy of 93.30% in classifying between malignant and benign tumor among the adult patients from the oncology department (Table 12), suggesting that our AI model is useful towards assisting physicians in the diagnosis process.

TABLE 9A
End-To-End Model Performance in Organ-System Based
Diagnostic Classifications of Adult Diseases

| | GMU 1 | | GMU2 |
|---|---|---|---|
| Adult AI Model | Train set | Test set | Test set |
| Tumor or cancer disease | 99.40% | 93.29% | 89.79% |
| Respiratory disease | 99.61% | 97.84% | 97.11% |
| Cardiovascular disease | 99.80% | 97.36% | 96.54% |
| Gynecological and Obstetric disease | 99.83% | 97.48% | 84.68% |
| Gastrointestinal disease | 99.83% | 97.00% | 95.81% |
| Neuropsychiatric disease | 99.81% | 93.17% | 97.17% |
| Urological disease | 99.95% | 97.84% | 94.11% |
| Orthopedic disease | 99.96% | 97.24% | 96.38% |
| Endocrinologic and metabolic disease | 99.93% | 96.16% | 96.93% |
| Ear-Nose-Throat disease | 100.00% | 97.12% | 88.05% |
| Nephrological disease | 100.00% | 94.12% | 96.18% |
| Ophthalmological disease | 99.96% | 97.60% | 81.39% |
| Average | 99.63% | 96.35% | 94.31% |

TABLE 9B
End-To-End Model Performance in Organ-System Based
Diagnostic Classifications of Pediatric Diseases

| | GWCMC1 | | GWCMC2 |
|---|---|---|---|
| Pediatric AI Model | Train set | Test set | Test set |
| Respiratory disease | 87.54% | 88.50% | 80.00% |
| Ear-Nose-Throat disease | 85.74% | 83.50% | 79.10% |
| Gastrointestinal disease | 97.24% | 96.80% | 95.30% |
| General Systemic disease | 93.46% | 92.10% | 88.70% |
| Neuropsychiatric disease | 99.34% | 97.80% | 97.40% |
| Urological disease | 99.10% | 92.40% | 81.20% |
| Average | 88.38% | 91.85% | 86.95% |

TABLE 10A
End-To-End Model Performance in Classifying
Upper vs Lower Respiratory Diseases in Adults

| | |
|---|---|
| Adult Upper vs. Lower | GMU 1 |

| Respiratory Systems | Train | Test |
|---|---|---|
| Upper Respiratory | 95.73% | 88.60% |
| Lower Respiratory | 93.74% | 94.00% |
| Average | 94.74% | 91.30% |

TABLE 10B
End-To-End Model Performance in Classifying Upper
vs Lower Respiratory Diseases in Pediatrics

| | |
|---|---|
| Pediatric Upper vs. Lower | GWCMC1 |

| Respiratory Systems | Train | Test |
|---|---|---|
| Upper Respiratory | 89.89% | 88.23% |
| Lower Respiratory | 87.25% | 86.01% |
| Average | 88.57% | 87.12% |

TABLE 11A
End-To-End Model Performance in Diagnosing
Common Pediatric Upper Respiratory Diseases

| Common Pediatric Upper | |
|---|---|
| Respiratory Diseases | Model Performance |
| Acute Upper Respiratory Infection | 93.90% |
| Sinusitis | 96.30% |
| Acute Laryngitis | 88.10% |
| Upper Tract Cough Syndrome | 90.70% |
| Overall Accuracy | 92.25% |

TABLE 11B
End-To-End Model Performance in Diagnosing
Common Pediatric Lower Respiratory Diseases

| Common Pediatric Lower | |
|---|---|
| Respiratory Diseases | Model Performance |
| Bronchitis | 81.30% |
| Pneumonia | 76.80% |
| Tracheitis | 90.40% |
| Asthma | 90.90% |
| Overall Accuracy: | 84.85% |

TABLE 12
Model Performance in Diagnosing Malignant vs. Benign Tumors

| | |
|---|---|
| Malignant vs. Benign | GMU 1 |

| Tumor Comparisons | Train set | Test set |
|---|---|---|
| Malignant Tumors | 95.51% | 95.20% |
| Benign Tumors | 96.56% | 91.40% |
| Average | 95.60% | 93.30% |

Validation of the AI Framework in Independent Adult and Pediatric Cohorts

External validations achieved comparable accuracies to internal validations thus confirming the diagnostic ability of the AI model. In diagnosing common disease categories, average diagnostic efficiency for adults was 94.31% and ranged from 81.39% (Ophthalmologic diseases) to 97.17% (Neuropsychiatric disease) in GMU2 external validation tests (FIG. 7A and Table 9A). The AUC of the micro-average ROC for adult classifications was 0.993 (FIG. 7B). Average diagnostic efficiency for pediatrics was 86.95% and ranged from 79.10% (Ear-Nose-Throat diseases) to 97.40% (Neuropsychiatric diseases) in the GWCMC2 external validation test (FIG. 7C and Table 9B). The AUC of the micro-average ROC for pediatric classifications was 0.983 (FIG. 7D).

Results of Error Analysis

We sought to characterize the cases misclassified by the end-to-end AI model by comparing occurrences of key discriminating words and phrases that led to a misdiagnosed prediction for the adult population. We analyzed the clinical document text to extract keywords for each common condition by evaluating the term-frequency-inverse-term-frequency (TF-IDF) scores (cite??) for each keyword within documents of each common condition diagnosis and across all conditions. The evaluation was done independently of the diagnosis model and its diagnosis. A total of 3,679 keywords were evaluated. Among those keywords with top TF-IDF scores, a physician manually selected an average of 13.83 keywords for each of the 12 common adult conditions that are uniquely distinctive in each of the common conditions (Table 13). From these selected keywords, we analyzed the misclassified clinical documents by our end-to-end AI model by a set of inclusion criteria to check if they contain sufficient information regarding ground-truth condition compared to the model diagnosed condition. A document was marked as containing insufficient or ambiguous information for the diagnosis if they satisfy one of the inclusion criteria (see Methods). 91.78% (335/365) of the misclassified documents were marked (Table 13). The analysis shows that misclassified EHRs by the framework are mostly due to either ambiguous or missing information related to ground-truth diagnosis conditions.

Performance Comparison Between the End-to-End Approach and the Hierarchical Diagnosis Approach

We previously developed an AI model to generate diagnoses in pediatric patients. This previous model followed a query-answer based schema curated by physicians to replicate clinical settings. Free text was extracted from EHRs to create clinical features or “answers” that were then manually mapped to hypothetical clinical queries following a hierarchical approach. These pairs were then fed through an attention-based LSTM using Tensorflow (Google Brain). The model was trained with 200,000 steps and achieved high accuracies, yet required extensive labeling of ground truth clinical features for sufficient training. The current model employs an end-to-end approach that negates the need labor-intensive labeling of ground truth clinical features. Here we compared the results from the previous AI model to the current end-to-end AI model in a common task of distinguishing upper vs. lower respiratory diseases, and found the results to be nearly identical (FIG. 8A-B, Table 14A). When evaluating each model's precision in diagnosing common disease phenotypes, the accuracy of the end-to-end AI model was slightly higher than the traditional model using expert-annotated clinical features. Average diagnostic efficiency in diagnosing common pediatric upper respiratory diseases was 89.43% compared to the current model's 92.25% accuracy (FIG. 8C-D, Table 14B). Average diagnostic efficiency in diagnosing common pediatric lower respiratory diseases was 83.40% compared to the current model's 84.85% accuracy (FIG. 8E-F, Table 14C). This suggested that given sufficient data, the end-to-end AI model may learn clinical features implicitly without extensive labeling efforts.

TABLE 14A
Traditional Schema vs. Current End-To-End Approach
in Classifying Upper and Lower Respiratory Diseases

| Upper vs. Lower Respiratory | Previous | Current |
|---|---|---|
| Systems: Traditional Schema vs. | Schema | End-To-End |
| Current End-To-End Approach | Model | Approach |
| Upper Respiratory | 88.00% | 88.00% |
| Lower Respiratory | 87.00% | 86.00% |

TABLE 14B
Traditional Schema vs. Current End-To-End Approach
in Diagnosing Common Upper Respiratory Diseases

| Common Pediatric Upper Respiratory | Previous | Current |
|---|---|---|
| Diseases: Traditional Schema vs. | Schema | End-To-End |
| Current End-To-End Approach | Model | Approach |
| Acute Upper Respiratory Infection | 93.20% | 93.90% |
| Sinusitis | 93.50% | 96.30% |
| Acute Laryngitis | 84.10% | 88.10% |
| Upper Tract Cough Syndrome | 86.90% | 90.70% |
| Overall Accuracy | 89.43% | 92.25% |

TABLE 14C
Traditional Schema vs. Current End-To-End Approach
in Diagnosing Common Lower Respiratory Diseases

| Common Lower Respiratory | Previous | Current |
|---|---|---|
| Diseases: Traditional Schema vs. | Schema | End-To-End |
| Current End-To-End Approach | Model | Approach |
| Bronchitis | 83.70% | 81.30% |
| Pneumonia | 74.20% | 76.80% |
| Tracheitis | 89.20% | 90.40% |
| Asthma | 86.50% | 90.90% |
| Overall Accuracy: | 83.40% | 84.85% |

Performance Comparison Between AI and Human Physicians

We further compared the diagnostic efficiencies between the AI model and physicians with variable levels of experience. The same internal validation test for adult patients (GMU1) consisting of 10,009 records was divided between a total of ten physicians and surgeons (three residents, four junior physicians; three chief physicians). Physicians reviewed corresponding medical records and proposed diagnoses that were then compared to original ground-truth diagnoses. These results were compared with the AI's performance in adult diseases. The physicians achieved an overall F-score average of 88.13% (range: 86.08% to 92.40%). Residents and Junior physicians achieved an overall F-score average of 86.66%; chief surgeons achieved an overall F-score average of 91.59%; the AI model achieved an overall F-score average of 95.98% (Table 15). Across the twelve major disease classification categories, the AI model outperformed physicians in every disease category with the exception of ophthalmological diseases; physicians correctly classified ophthalmological disease 98.17% of the time compared to the AI model's 97.60% accuracy. When evaluating 11,926 pediatric records, model performance was comparable to pediatricians. Junior physicians achieved an overall F-score average of 83.9%; chief surgeons achieved an overall F-score average of 91.6%; the AI model achieved an overall F-score average of 87.2%. Thus, the AI model outperformed junior physicians across the twelve disease classifications.

TABLE 15A
Physician vs. AI Model Comparison. We used F1-score to evaluate the diagnosis performance across different diagnosis groups
(rows) between our model, and three resident physician groups, four junior physician groups and three senior physician groups
(columns, see method section for description). We observed that our model performed better than all physician groups.

| Adult | | Junior | Junior | Junior | Junior | Senior | Senior | Senior | Resident | Resident | Resident | Overall |
|---|---|---|---|---|---|---|---|---|---|---|---|---|
| Disease | AI | Physi- | Physi- | Physi- | Physi- | Physi- | Physi- | Physi- | Physi- | Physi- | Physi- | Physi- |
| category | Model | cian #1 | cian #2 | cian #3 | cian #4 | cian #1 | cian #2 | cian #3 | cian #1 | cian #2 | cian #3 | cian |
| Tumor or | 93.29% | 81.73% | 85.23% | 83.67% | 82.78% | 88.65% | 87.36% | 87.67% | 86.43% | 81.37% | 85.16% | 85.01% |
| cancer disease | | | | | | | | | | | | |
| Respiratory | 97.84% | 82.56% | 83.33% | 82.61% | 86.87% | 93.42% | 91.95% | 86.59% | 82.46% | 84.81% | 89.54% | 86.41% |
| disease | | | | | | | | | | | | |
| Cardiovascular | 97.36% | 86.53% | 84.76% | 89.19% | 85.53% | 94.59% | 94.90% | 86.41% | 88.89% | 95.89% | 85.53% | 89.22% |
| disease | | | | | | | | | | | | |
| Gynecological | 93.01% | 96.32% | 94.00% | 92.42% | 88.10% | 92.86% | 98.70% | 96.39% | 91.67% | 80.65% | 86.67% | 91.78% |
| and Obstetric | | | | | | | | | | | | |
| disease | | | | | | | | | | | | |
| Gastrointestinal | 97.00% | 82.98% | 91.36% | 87.50% | 85.06% | 91.43% | 94.74% | 89.42% | 91.30% | 91.30% | 93.33% | 89.84% |
| disease | | | | | | | | | | | | |
| Neuropsychiatric | 93.17% | 85.09% | 84.86% | 85.71% | 81.78% | 85.78% | 87.09% | 86.89% | 83.56% | 83/54% | 83.78% | 84.95% |
| disease | | | | | | | | | | | | |
| Urological | 97.84% | 83.33% | 83.33% | 98.00% | 85.13% | 89.78% | 92.32% | 91.67% | 89.67% | 87.55% | 89.19% | 89.00% |
| disease | | | | | | | | | | | | |
| Orthopedic | 97.24% | 87.50% | 91.65% | 91.03% | 83.34% | 92.65% | 93.46% | 92.33% | 89.33% | 82.67% | 73.91% | 87.79% |
| disease | | | | | | | | | | | | |
| Endocrinologic | 96.16% | 83.45% | 81.37% | 82.58% | 83.46% | 89.82% | 90.21% | 89.37% | 84.29% | 83.36% | 81.97% | 84.99% |
| and metabolic | | | | | | | | | | | | |
| disease | | | | | | | | | | | | |
| Ear-Nose- | 97.12% | 87.25% | 83.16% | 82.36% | 83.45% | 92.34% | 91.78% | 92.08% | 81.67% | 79.79% | 83.98% | 85.79% |
| Throat disease | | | | | | | | | | | | |
| Nephrological | 94.12% | 81.68% | 82.69% | 84.08% | 82.67% | 91.25% | 89.25% | 88.98% | 80.43% | 81.58% | 83.65% | 84.63% |
| disease | | | | | | | | | | | | |
| Ophthalmological | 97.60% | 97.50% | 99.17% | 99.00% | 98.00% | 99.26% | 97.00% | 98.76% | 97.00% | 97.96% | 98.01% | 98.17% |
| disease | | | | | | | | | | | | |
| Average F-score | 95.98% | 86.33% | 87.08% | 88.18% | 85.51% | 91.82% | 92.40% | 90.55% | 87.32% | 86.08% | 86.23% | 88.13% |

TABLE 15B
Illustration of diagnostic performance between our AI model and
pediatricians We used F1-score to evaluate the diagnosis performance
across different diagnosis groups (rows) between our model, and
two junior physician groups and three senior physician groups
(columns, see method section for description). We observed that
our model performed better than junior physician groups but slightly
worse than three experienced physician groups.

| | | Physicians | | | | |
|---|---|---|---|---|---|---|
| | | Junior | Junior | Senior | Senior | Senior |
| | | Physi- | Physi- | Physi- | Physi- | Physi- |
| | | cian | cian | cian | cian | cian |
| | AI | group | group | group | group | group |
| | model | #1 | #2 | #3 | #4 | #5 |
| Asthma | 84.0% | 80.1% | 83.7% | 90.4% | 89.0% | 93.5% |
| Encephalitis | 81.0% | 94.7% | 96.1% | 95.0% | 95.9% | 96.5% |
| GI | 86.5% | 81.8% | 87.2% | 85.4% | 89.6% | 89.3% |
| Group: | 78.6% | 80.8% | 73.0% | 87.9% | 94.0% | 94.3% |
| “Acute | | | | | | |
| laryngitis” | | | | | | |
| Group: | 88.8% | 82.9% | 76.7% | 94.6% | 95.2% | 97.2% |
| “Pneumonia” | | | | | | |
| Group: | 93.2% | 83.9% | 79.7% | 89.6% | 87.3% | 87.0% |
| “Sinusitis” | | | | | | |
| Lower | 80.3% | 80.3% | 81.5% | 91.0% | 90.3% | 93.5% |
| Respiratory | | | | | | |
| Mouth-related | 89.7% | 81.8% | 87.2% | 85.4% | 89.6% | 89.3% |
| Diseases | | | | | | |
| Neuro-Psych | 94.0% | 92.5% | 96.3% | 96.0% | 96.2% | 90.6% |
| Respiratory | 89.4% | 80.8% | 76.9% | 89.0% | 90.7% | 91.7% |
| Systemic- | 91.2% | 87.9% | 90.7% | 95.2% | 90.7% | 94.4% |
| Generalized | | | | | | |
| Upper | 89.1% | 81.7% | 75.4% | 88.4% | 91.6% | 91.6% |
| respiratory | | | | | | |
| Average F- | 87.2% | 84.1% | 83.7% | 90.7% | 91.7% | 92.3% |
| score | | | | | | |

AI can Provide Improvement of Hospital Management

We next conducted a study to address hospital management efficiency. We compared times of visits, costs, and admission rate between two groups where AI and physician diagnoses were concordant versus AI and physician diagnoses were discordant among top frequent disease categories. We showed there are marked differences in these two groups. In general, patients in the discordant groups have more visits, higher costs, and higher admission rates (Table 16), indicating beneficial effect of AI in assisting hospital management.

TABLE 16
AI can improve hospital management efficiencies. We analyzed 7 diseases categories which
constituted the most frequent hospital visit. Match: diagnosis is concordant between
AI and pediatricians; Mismatch, diagnosis is discordant between AI and pediatricians.

| | | | | | Cost in | | | | |
|---|---|---|---|---|---|---|---|---|---|
| | | | | | outpatient | | Number of | | |
| Disease | | Number of | Visit times | | visit (RMB) | | inpatient | admission | Concordance |
| category | Group | records | Mean | STD | Mean | STD | Admissions | rate | Rate |
| Infectious | Match | 328 | 4.05 | 2.02 | 1313.29 | 847.12 | 48 | 14.63% | 76.64% |
| mononucleosis | Mismatch | 100 | 4.85 | 2.43 | 1502.79 | 1129.8 | 36 | 36.00% | |
| Mycoplasma | Match | 296 | 2.84 | 1.92 | 837.42 | 658.15 | 2 | 0.68% | 64.91% |
| infection | Mismatch | 160 | 3.13 | 2.23 | 1024.04 | 853.71 | 1 | 0.63% | |
| Acute | Match | 1297 | 2.23 | 1.81 | 495.78 | 480.09 | 31 | 2.39% | 77.48% |
| tonsillitis, | Mismatch | 377 | 2.57 | 1.92 | 637.09 | 543.24 | 13 | 3.45% | |
| Acute | Match | 2134 | 2.4 | 1.95 | 530 | 474.78 | 110 | 5.15% | 75.54% |
| laryngitis | Mismatch | 691 | 3.88 | 2.74 | 949.25 | 761.84 | 53 | 7.67% | |
| Influenza flu | Match | 401 | 1.97 | 1.47 | 416.48 | 430.48 | 24 | 5.99% | 76.09% |
| | Mismatch | 126 | 2.29 | 1.5 | 615.23 | 562.56 | 19 | 15.08% | |
| Enteroviral | Match | 1440 | 2.28 | 1.67 | 478.45 | 425.52 | 50 | 3.47% | 88.40% |
| vesicular | Mismatch | 189 | 2.67 | 2.17 | 586.02 | 436.96 | 15 | 7.94% | |
| stomatitis | | | | | | | | | |
| with exanthem | | | | | | | | | |
| asthmatic | Match | 1047 | 2.64 | 1.87 | 792.41 | 652.22 | 13 | 1.24% | 68.16% |
| bronchitis | Mismatch | 489 | 3.11 | 2.13 | 976.13 | 824.16 | 28 | 5.73% | |

Identification of Common Features Driving Diagnostic Prediction

In an effort to build a system that guides patients towards a diagnosis, the key driving words and the coding parameters (i.e. binary or categorical classification) that lead to an accurate diagnosis prediction were identified.

First, it was determined that a short chief complaint statement is sufficient for the framework to accurately identify the diagnosis of a patient, suggesting that the framework can potentially be built into a text-based automatic triage system that can provide initial evaluation of these common diseases.

Given the keywords identified from the word segmentation method applied to the available clinical documents, the term-frequency-inverse-term-frequency (TF-IDF) scores for each keyword was evaluated within documents of each common condition diagnosis and across all conditions. The evaluation was done independently of the diagnosis model and its diagnosis. A total of 3,679 keywords were evaluated (Table 13). Among those keywords with top TF-IDF scores, a physician manually selected an average of 13.83 keywords for each of the 12 common adult conditions that are uniquely distinctive in each of the common conditions (Table 13).

From these selected keywords, we analyzed the misclassified clinical documents by our end-to-end AI model by a set of inclusion criteria to check if they contain sufficient information regarding ground-truth condition compared to the model diagnosed condition. A document was marked as containing insufficient or ambiguous information for the diagnosis if they satisfy one of the inclusion criteria (see Methods). 91.78% (335/365) of the misclassified documents were marked. The analysis shows that misclassified EHRs by the framework are mostly due to either ambiguous or missing information related to ground-truth diagnosis conditions.

Discussion

Supervised machine learning is highly applicable and currently under-utilized in the medical field. Whereas previous learning systems required training parameters in a monotonous, step-by-step order, end-to-end learning trains parameters in a simultaneous manner that automatically maps the relationship between inputs and outputs. As shown, our end-to-end approach achieved comparable results to the traditional model in diagnosing specific respiratory diseases without requiring labor-intensive annotation of ground truth clinical features. As a means to access a multitude of variables provided in the physician consultation notes, we used an end-to-end approach to link free text from EHRs to accurately predict primary disease diagnosis via a NLP-based deep learning hybrid. For training purposes, annotations from expert physicians and informatics researchers were processed through an AI model as a means to extract important clinical features. This AI model was then applied to physician notes from over 2.61 million encounters across several major referral hospitals in China to extract meaningful clinical features into a deep learning classifier. Our model achieved a high level of accuracy in disease classification and predicting disease diagnosis across all common adult and pediatric conditions when compared to the original assessment and covers a wide range of disease categories. Furthermore, error analysis showed that records misclassified by our AI system were mostly due to missing or ambiguous information from the records. Therefore, discrepancies between AI and final diagnosis may suggest the need to improve the reporting quality of records in EHR.

One of the major challenges in healthcare across the globe is the increasing patient population and limited medical resources. In the top 18 countries serving 50% of the world's population, the mean consultation time is five minutes. In Bangladesh, for instance, the average consultation time is 48 seconds. Research has shown that a human's processing capacity often plateaus around four variables, therefore to obtain the relevant clinical information from the patient and deduce a diagnosis based on a number of variables within a few minutes is error prone. Deep learning can easily extract relationships between hundreds of variables across multiple dimensions within a relatively short time frame. When comparing average diagnostic efficiencies between our model and physicians, our model outperformed disease classifications in all categories with the exception of ophthalmological cases. In classifying diseases such as endocrinology and nephrology, model was able to better identify these conditions compared to physicians with accuracies of 38.75% and 41.06% respectively, demonstrating its efficacy as a diagnostic tool in clinical evaluation. Furthermore, our AI model showed high efficiency in diagnosing specific common diseases across a range of disease categories which may better serve hospital management by accurately triaging patients. For instance, by implementing an AI-assisted triaging system, patients who are diagnosed with more urgent or life-threatening conditions could be prioritized over those with relatively benign conditions. Under these circumstances, more hospital time and/or resources could be allocated to patients with greater or more urgent medical need compared to those who could bypass urgent physician evaluation and be referred for routine outpatient assessment.

Error analysis showed that records misclassified by the AI system are mostly due to missing or ambiguous information from the records. Therefore, discrepancies between AI and final diagnosis may suggest the need to improve the reporting quality of records in EHR. By comparing visits, costs, and admission rate, hospital stay duration, admission rate between AI and physician diagnosis-concordant group versus AI and physician diagnosis-discordant group among top disease categories, it was shown that the AI system can provide beneficial effect of AI in assisting hospital management, and reducing complications.

AI implementation, however, should not negate medicine's need for a compassionate hand, but rather augment the services provided to our patients. Disease is not biased, so neither should healthcare. However, often times past experiences may cause a physician to inaccurately place more emphasis on certain features than others leading to misdiagnosis, especially those pertaining to rare diseases. AI utilizes data from millions of patients across the globe and is trained on a wide array of outcomes that many physicians may not experience in their relative expertise. AI could serve the physician as a knowledgeable, unbiased assistant in diagnosing diseases they may often be overlooked. Furthermore, AI can take into account features that may be considered insignificant in clinical settings, such as certain socioeconomic factors, race, etc., which could make AI particularly useful in the applications of epidemiology.

In conclusion, the hybrid NLP deep learning model was able to accurately assess primary disease diagnosis across a range of organ systems and subsystems. The potential benefits of the model's application to hospital management efficiencies by reducing costs and hospital stay was shown. This system shows great potential in triaging patients in areas where healthcare providers are in relative shortage compared to the overall population, such as China or Bangladesh, and in providing clinical aide to patients in rural environments where physicians are not as easily accessible.

For example, our NLP deep learning model was able to accurately classify presenting diseases into adult and pediatric ICD-10 categories, with the ability to further diagnose specific disease conditions. The model outperformed physicians in almost all categories in terms of diagnostic efficiency, therefore demonstrating its potential utility as a diagnostic aide that could be used to triage patients in areas of healthcare resource shortage or provide a resource for patients in environments where access to care may be limited.

Methods

Data Collection

A retrospective study was conducted on 2,612,114 EHRs (380,665 adult; 2,231,449 pediatric) from 1,085,795 patients (223,907 adult, 861,888 pediatric). The First Affiliated Hospital of Guangzhou Medical University (GMU 1), a major academic tertiary medical referral center, provided 186,745 adult patients with 333,672 EHRs for training and internal validation purposes. Guangzhou Women and Children's Medical Center (GWCMC1), a major academic pediatric medical referral center, provided 552,789 outpatient and inpatient pediatric visits consisting of 1,516,458 EHRs for training and internal validation purposes. The Second Affiliated Hospital of Guangzhou Medical University (GMU 2), provided 37,162 patients consisting of 46,993 EHRs for external validation purposes in adults. A separate cohort of pediatric data from Guangzhou Women and Children's Medical Center (GWCMC2) was collected over later time points which did not overlap with those used in the machine learning. This data provided 339,099 patients with 714,991 EHRs for external validation in pediatrics. These records encompassed physician encounters for pediatric and adult patients presenting to these medical institutions from January 2016 to October 2018. The study was approved by the First Affiliated Hospital of Guangzhou Medical University, the Second affiliated Hospital of Guangzhou Medical University, and Guangzhou Women and Children's Medical Center. This study complied with the Declaration of Helsinki and institutional review board and ethics committee. For all encounters, physicians classified primary diagnosis through the use of the International Classification of Disease ICD-10 codes. Twelve ICD 10 codes encompassed adult diseases while 6 ICD 10 codes encompassed common pediatric diseases. Certain disease categories, such as gynecological/obstetric and cardiovascular diseases, were considered inapplicable to include for pediatric analysis and were therefore excluded. All disease categories provide a wide range of pathology across adult and pediatric cohorts.

The End-to-End AI Model Framework

The diagnostic model utilized free-text descriptions available in EHRs generated from Zesing Electronic Medical Records. The model reviewed the following three parameters per patient visit; chief complaints, history of present illness, and picture archiving and communication system (PACS) reports. Given that all EHRs were obtained from Chinese cohorts, text segmentation was essential in Chinese NLP due to the lack of spaces that separate meaningful units of text. As such, a comprehensive Chinese medical dictionary 10 and Jieba, a widely used open-source general-purpose Chinese word/phrase segmentation software, were customized and applied to each record as a means to extract text containing relevant medical information (Supplementary FIG. 1). These extracted words were then fed into a word embedding layer to convert text into 1×100 vector dimensions. Vectors were then fed into a bi-directional long-short term memory (LSTM) recurrent neural network using PyTorch's default configuration that comprises 256 hidden units for each of the two layers. The model learns word embedding vectors for all 552,700 words and phrases in the vocabulary and all the weights in the bidirectional LSTM. The learning rate was set to default 0.001 in all of our model training processes. The output vectors of LSTM of each direction are concatenated and fed into a fully-connected SoftMax layer that computes a score for each diagnostic class. The class with the highest score is considered the model's diagnosis (FIG. 1). The model was trained end-to-end to obtain optimal model parameters for all layers without any feature engineering other than the initial word segmentation. No labor-intensive labeling of clinical features was necessary to train the model.

Error Analysis

The 365 adult clinical records that were misclassified into an incorrect diagnosis among one of the twelve adult common conditions were considered. The records with the keywords that were identified for each condition were compared. A record was considered to contain missing information or ambiguous if it satisfies one of the following inclusion criteria:

No ground truth condition keywords.

More keywords for the predicted condition than keywords for the ground-truth condition.

Less than five keywords for the ground-truth condition.

Less than ten keywords from either the ground-truth or predicted conditions.

More than one chief complaint section.

More than one history of present illness section.

Next, a similar error analysis was performed for the 1,095 adult clinical records that were misclassified when the model took only the chief complaints as the input. Since a chief complaint is short, only the first two criteria were considered in this case.

Comparative Performance Between Our AI System and Human Physicians

We conducted a comparison study between our AI system versus human physicians. Free text, patient ID, and date of evaluation from 10,008 EHRs from GMU 1 internal validation test set were randomly sorted and equally divided between ten family medicine/general practitioners and chief physicians to manually label disease diagnosis. Two resident physicians and one resident surgeon with 1-2 years of practice experience, three junior physicians and one junior surgeon with 5-7 years of practice experience, and three chief surgeons with 8-10 years of practice experience made up the conglomerate of the practitioners. We evaluated the diagnostic performance of each physician group in each of top 12 diagnosis categories using an F1-score.

1. A method for providing a medical diagnosis, comprising:
a) obtaining medical data;
b) using a natural language processing (NLP) information extraction model to extract and annotate clinical features from the medical data; and
c) analyzing at least one of the clinical features with a disease prediction classifier to generate a classification of a disease or disorder, the classification having a sensitivity of at least 80%.

2. The method of claim 1, wherein the NLP information extraction model comprises a deep learning procedure.

3. The method of claim 1, wherein the NLP information extraction model utilizes a standard lexicon comprising keywords representative of assertion classes; or wherein the NLP information extraction model utilizes a plurality of schema, each schema comprising a feature name, anatomical location, and value.

4. (canceled)

5. The method of claim 4, wherein the plurality of schema comprises at least one of history of present illness, physical examination, laboratory test, radiology report, and chief complaint.

6. The method of claim 1, further comprising tokenizing the medical data for processing by the NLP information extraction model.

7. (canceled)

8. The method of claim 1, wherein the classification has a specificity of at least 80%, or the classification has an F1 score of at least 80%.

9. (canceled)

10. (canceled)

11. The method of claim 1, wherein the disease prediction classifier comprises a logistic regression classifier or a decision tree.

12. (canceled)

13. The method of claim 1, wherein the classification differentiates between a serious and a non-serious condition.

14. The method of claim 1, wherein the classification comprises at least two levels of categorization.

15. The method of claim 1, wherein the classification comprises a first level category indicative of an organ system, and optionally further comprises a second level indicative of a subcategory of the organ system.

16. (canceled)

17. The method of claim 1, wherein the classification comprises a diagnostic hierarchy that categorizes the disease or disorder into a series of narrower categories.

18. The method of claim 17, wherein the classification comprises a categorization selected from the group consisting of respiratory diseases, genitourinary diseases, gastrointestinal diseases, neuropsychiatric diseases, and systemic generalized diseases.

19. The method of claim 18, wherein the classification further comprises a subcategorization of respiratory diseases into upper respiratory diseases and lower respiratory diseases.

20. The method of claim 19, wherein the classification further comprises a subcategorization of upper respiratory disease into acute upper respiratory disease, sinusitis, or acute laryngitis.

21. The method of claim 19, wherein the classification further comprises a subcategorization of lower respiratory disease into bronchitis, pneumonia, asthma, or acute tracheitis.

22. The method of claim 18, wherein the classification further comprises a subcategorization of gastrointestinal diseases into diarrhea, mouth-related diseases, or acute pharyngitis.

23. The method of claim 18, wherein the classification further comprises a subcategorization of neuropsychiatric diseases into tic disorder, attention-deficit hyperactivity disorder, bacterial meningitis, encephalitis, or convulsions.

24. The method of claim 18, wherein the classification further comprises a subcategorization of systemic generalized diseases into hand, foot and mouth disease, varicella without complication, influenza, infectious mononucleosis, sepsis, or exanthema subitum.

25. The method of claim 1, further comprising making a medical treatment recommendation based on the classification.

26. The method of claim 1, wherein the disease prediction classifier is trained using end-to-end deep learning.

27.-30. (canceled)
